(19) United States
(12) Patent Application Publication
(10) Pub. No.: US 2023/0303604 A1
(43) Pub. Date: Sep. 28, 2023

(54) METALLIC TRANS-(N-HETEROCYCLIC CARBENE)-AMINE-PLATINUM COMPLEXES AND USES THEREOF FOR TREATING CANCER

(71) Applicants: INSTITUT CURIE, Paris (FR); INSTITUT NATIONAL DE LA SANTE ET DE LA RECHERCHE MEDICALE, Paris (FR); CENTRE NATIONAL DE LA RECHERCHE SCIENTIFIQUE, Paris (FR); UNIVERSITE PARIS-SACLAY, Gif Sur Yvette (FR)

(72) Inventors: Angela MARINETTI, Gif Sur Yvette (FR); Jean-François BETZER, Gif Sur Yvette Cedex (FR); Sophie BOMBARD-CAUCAT, Orsay Cedex (FR); Deepanjan GHOSH, Paris (FR); Tao JIA, Chengdu, Sichuan (FR)

(21) Appl. No.: 18/249,766

(22) PCT Filed: Oct. 19, 2021
(86) PCT No.: PCT/EP2021/078966

§ 371 Date: Apr. 20, 2023

(30) Foreign Application Priority Data
Oct. 20, 2020 (EP) 20306246.8

(51) Int. Cl. C07F 15/00; A61P 35/00

(52) U.S. Cl. CPC C07F 15/0086; A61P 35/00

(57) ABSTRACT

The present invention concerns a compound of formula (I), in particular as radiosensitizer agent. The present invention also concerns these new compounds for use for treating cancer such as glioblastoma, lung cancer, or ovarian cancer, in particular in combination with radiotherapy or with an anticancer drug.

The present invention concerns new bimetallic trans-(N-Heterocyclic Carbene)-amine-Pt(II) complexes, and uses thereof for treating cancer in particular in combination with radiotherapy or in combination with radiotherapy and any anticancer drug. The present invention also concerns monometallic (Amine)Platinum(II) N-Heterocyclic Carbene complexes for treating cancer in particular in combination with radiotherapy or in combination with radiotherapy and any anticancer drug.

Combined therapy is often used in clinic due to the fact that some drugs can induce synergistic effects when used at their subtoxic concentrations, thereby reducing their secondary toxic effects. Radiotherapy, is one of the most used modality in clinic (in 50% of the cases) but suffers some limitations namely radioresistance of tumor cells and reactions in normal tissue around the tumor. These limitations can be overcome by the combination of radiotherapy with drugs that act as radiosensitizers. Indeed, this chemo-radiotherapy is a standard treatment for many different cancers such as ovarian, non-small cell lung cancer (NSCLC), glioblastoma, bladder, rectal, cervix, head and neck cancer . . . (Sharma, R. A., et al. (2016) Clinical development of new drug-radiotherapy combinations, Nature reviews. Clinical oncology 13, 627-642). Since radiotherapy is based on the induction of a large number of DNA damages that if not correctly repaired would induce cell death, combination therapy has been divided in several classes of agents that may impair the DNA repair or increase the DNA damages. Among them, DNA binding and damaging agents have emerged and the clinical use of the platinum complex cisplatin in combination with ionizing radiations encouraged the development of other metallic complexes and their assessment in potential radiosensitizing properties (Gill, M. R., and Vallis, K. A. (2019) Transition metal compounds as cancer radiosensitizers, Chem Soc Rev 48, 540-557). In contrast to their extensive research as possible chemotherapeutic agents, their possible use as radiosensitizer has been largely ignored. Moreover, the mechanism of radiosensitizing effect of cisplatin in cellular experiments remains controversial, since it depends on the cell lines and drug administration protocol (Wilson, G. D., Bentzen, S. M., and Harari, P. M. (2006) Biologic basis for combining drugs with radiation, Seminars in radiation oncology 16, 2-9). Even if its clinical effect seems more correlated to an additive than synergistic effect, patients treated with cisplatin-based chemotherapy and radiotherapy have longer survival times than those treated by each single treatment (Mierzwa, M. L., Nyati, M. K., Morgan, M. A., and Lawrence, T. S. (2010) Recent advances in combined modality therapy, The oncologist 15, 372-381). However, in advanced non-small cell lung cancer (NSCLC), whereas chemotherapy is the treatment of choice for locally, the overall survival rates remain weak in all but earliest stages of treatment (Baas, P., Belderbos, J. S., and van den Heuvel, M. (2011) Chemoradiation therapy in nonsmall cell lung cancer, Current opinion in oncology 23, 140-149; and Uyterlinde, W. (2016) Overcoming toxicity-challenges in chemoradiation for non-small cell lung cancer, Translational Lung Cancer Research 5, 239-243).

It is known that these DNA damaging agents also have detrimental effects in normal cells due to the fact that they target DNA, but their associated with irradiation at their subtoxic doses may lead to the emergence of new treatments. All together, these data show that developing, identifying and understanding the mechanism of action new drugs to increase radiosensitivity is an essential strategy in the treatment of some cancers.

In addition, cisplatin which is a metallic coordination compound used as chemotherapeutic drug for treatment of numerous cancers and effective against carcinomas, germ cell tumors lymphomas and sarcomas (Dasari, S., and Bernard Tchounwou, P. (2014) Cisplatin in cancer therapy: Molecular mechanisms of action, European Journal of Pharmacology 740, 364-378) generates crosslinks with the purine bases, causing DNA damage. However, its use in therapy may be limited due to: i) decrease in anticancer activity against certain cancers, ii) the phenomena of acquired resistance developed by many tumours and iii) numerous undesirable side effects due to a lack of selectivity. The search for new platinum complexes, with different DNA binding properties from those of cisplatin, would make it possible to overcome the resistance difficulties encountered and also to improve the pharmacological profile of these complexes. For instance, the platinum(II) complexes with trans configuration are known to form intrastrand bifunctional adducts between two guanines separated by a single nucleotide, GXG and interstrand adducts between the guanine and the cytosine facing it (Jung, Y., and Lippard, S. J. (2007) Direct cellular responses to platinum-induced DNA damage, Chem. Rev. 107, 1387-1407).

There is thus a need for platinum complexes, with different DNA binding properties from those of cisplatin, in order to overcome the resistance difficulties encountered and also to improve the pharmacological profile of these complexes.

The aim of the present invention is thus to provide a new series of organometallic with an unprecedented bi-metallic molecular scaffold displaying radiosensitizing properties.

The aim of the present invention is also to provide new platinum complexes with radiosensitizing properties.

Thus, the present invention relates to a mono- or bimetallic (Amine)Platinum(II) N-Heterocyclic Carbene complex having the following formula (I-1):

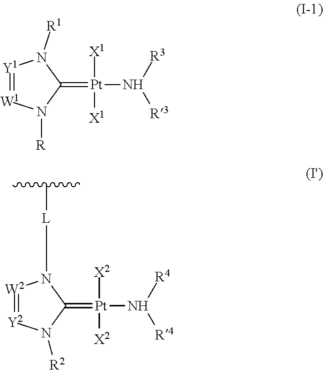

wherein:
R is a group having the below formula (I′):

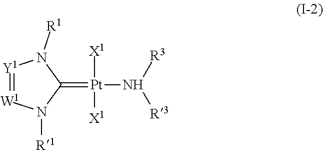

or R is selected from the group consisting of: a C₁-C₆ alkyl group, a C₃-C₆ cycloalkyl, a C₆-C₁₀ aryl, and a (C₆-C₁₀)aryl(C₁-C₆)alkyl group;
L is a linker selected from the group consisting of: a C₁-C₁₂ alkanediyl group, a phenylene-bis(alkanediyl) group, a biphenyldiyl-bis(alkanediyl) group, and an heteroarylidene-bis(alkanediyl) group, said alkanediyl, phenylene and heteroarylidene groups being possibly substituted with one or several substituents such as C₁-C₆ alkyl groups, C₅-C₁₀ aryl groups, heteroaryl, and (hetero)cycloalkyl groups,
   wherein said alkanediyl groups may be interrupted with one or several heteroatoms;
X¹ and X², identical or different, are selected from the group consisting of: iodide, bromide, chloride, and nitrato (ONO₂);
Y¹ and Y², identical or different, are either a C—R⁵ group or a N atom, R⁵ being selected from the group consisting of: H, a C₁-C₆ alkyl group, C₃-C₆ cycloalkyl group, and an optionally substituted phenyl group,
W¹ and W², identical or different, are either a C—R⁶ group or a N atom, R⁶ being selected from the group consisting of: H, a C₁-C₆ alkyl group, C₃-C₆ cycloalkyl group, and an optionally substituted phenyl group,
   or Y¹ and W¹ are tethered to form a cyclic unit, said tether being a C₃-C₆ alkanediyl chain with one or more heteroatoms or an unsaturated C₃-C₆ chain;
   or Y² and W² are tethered to form a cyclic unit, said tether being a C₃-C₆ alkanediyl chain with one or more heteroatoms or an unsaturated C₃-C₆ chain;
R¹ and R², identical or different, are selected from the group consisting of: a C₁-C₆ alkyl group, a C₃-C₆ cycloalkyl, a C₆-C₁₀ aryl, and a (C₆-C₁₀)aryl(C₁-C₆)alkyl group, said C₁-C₆ alkyl group being optionally substituted with an hydroxyl group, or substituted with a —C(═O )—NH—(C₆-C₁₀)aryl, preferably with a —C(═O)—NH-phenyl group,
   or R¹ and Y¹ can also be tethered to form a cyclic unit with the nitrogen atom bearing R¹, said tether being a C₃-C₄ alkanediyl chain or an unsaturated C₃-C₆ alkanediyl group, an heteroalkanediyl with one or more N atoms, wherein the carbons of the chain may also be part of a carbonyl group,
   or R² and Y² can also be tethered to form a cyclic unit with the nitrogen atom bearing R², said tether being a C₃-C₄ alkanediyl chain or an unsaturated C₃-C₆ alkanediyl group, an heteroalkanediyl with one or more N atoms, wherein the carbons of the chain may also be part of a carbonyl group,
R³ and R′³ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R³ and R′³ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms, and
R⁴ and R′⁴ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R⁴ and R′⁴ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms,
   for use as radiosensitizer.

The present invention also relates to a monometallic (Amine)Platinum(II) N-Heterocyclic Carbene complex having the following formula (I-2):

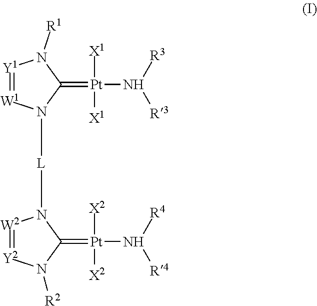

wherein:
R′¹ is selected from the group consisting of: a C₁-C₆ alkyl group, a C₃-C₆ cycloalkyl, a C₆-C₁₀ aryl, and a (C₆-C₁₀)aryl(C₁-C₆)alkyl group;
X¹ is selected from the group consisting of: iodide, bromide, chloride, and nitrato (ONO₂);
Y¹ is either a C—R⁵ group or a N atom, R⁵ being selected from the group consisting of: H, a C₁-C₆ alkyl group, C₃-C₆ cycloalkyl group, and an optionally substituted phenyl group,
W¹ is either a C—R⁶ group or a N atom, R⁶ being selected from the group consisting of: H, a C₁-C₆ alkyl group, C₃-C₆ cycloalkyl group, and an optionally substituted phenyl group,
   or Y¹ and W¹ are tethered to form a cyclic unit, said tether being a C₃-C₆ alkanediyl chain with one or more heteroatoms or an unsaturated C₃-C₆ chain;
R¹ is selected from the group consisting of: a C₁-C₆ alkyl group, a C₃-C₆ cycloalkyl, a C₆-C₁₀ aryl, and a (C₆-C₁₀)aryl(C₁-C₆)alkyl group,
   or R¹ and Y¹ can also be tethered to form a cyclic unit with the nitrogen atom bearing R¹, said tether being a C₃-C₄ alkanediyl chain or an unsaturated C₃-C₆ alkanediyl group, an heteroalkanediyl with one or more N atoms, wherein the carbons of the chain may also be part of a carbonyl group,
R³ and R′³ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R³ and R′³ together form a C₃-C₄ alkanediyl chain, an unsaturated C₃-C₆ alkanediyl group, or an heteroalkanediyl group with O or N atoms,
   for use as radiosensitizer.

According to an embodiment, in formula (I-2), L is a C₁-C₁₂ alkanediyl group, and more preferably a C₂-C₁₂ alkanediyl group.

The present invention also relates to a bimetallic (Amine)Platinum(II) N-Heterocyclic Carbene complex having the following formula (I):

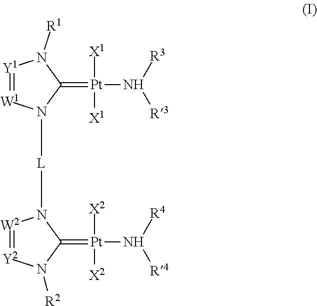

wherein:
L is a linker selected from the group consisting of: a C₁-C₁₂ alkanediyl group, a phenylene-bis(alkanediyl) group, a biphenyldiyl-bis(alkanediyl) group, and an heteroarylidene-bis(alkanediyl) group, said alkanediyl, phenylene and heteroarylidene groups being possibly substituted with one or several substituents such as C₁-C₆ alkyl groups, C₅-C₁₀ aryl groups, heteroaryl, and (hetero)cycloalkyl groups,
   wherein said alkanediyl groups may be interrupted with one or several heteroatoms;
X¹ and X², identical or different, are selected from the group consisting of: iodide, bromide, chloride, and nitrato (ONO₂);
Y¹ and Y², identical or different, are either a C—R⁵ group or a N atom, R⁵ being selected from the group consisting of: H, a C₁-C₆ alkyl group, C₃-C₆ cycloalkyl group, and an optionally substituted phenyl group,
W¹ and W², identical or different, are either a C—R⁶ group or a N atom, R⁶ being selected from the group consisting of: H, a C₁-C₆ alkyl group, C₃-C₆ cycloalkyl group, and an optionally substituted phenyl group,
   or Y¹ and W¹ are tethered to form a cyclic unit, said tether being a C₃-C₆ alkanediyl chain with one or more heteroatoms or an unsaturated C₃-C₆ chain;
   or Y² and W² are tethered to form a cyclic unit, said tether being a C₃-C₆ alkanediyl chain with one or more heteroatoms or an unsaturated C₃-C₆ chain;
R¹ and R², identical or different, are selected from the group consisting of: a C₁-C₆ alkyl group, a C₃-C₆ cycloalkyl, a C₆-C₁₀ aryl, and a (C₆-C₁₀)aryl(C₁-C₆)alkyl group, said C₁-C₆ alkyl group being optionally substituted with an hydroxyl group, or substituted with a —C(═O)—NH—(C₆-C₁₀)aryl, preferably with a —C(═O)—NH-phenyl group,
   or R¹ and Y¹ can also be tethered to form a cyclic unit with the nitrogen atom bearing R¹, said tether being a C₃-C₄ alkanediyl chain or an unsaturated C₃-C₆ alkanediyl group, an heteroalkanediyl with one or more N atoms, wherein the carbons of the chain may also be part of a carbonyl group,
   or R² and Y² can also be tethered to form a cyclic unit with the nitrogen atom bearing R², said tether being a C₃-C₄ alkanediyl chain or an unsaturated C₃-C₆ alkanediyl group, an heteroalkanediyl with one or more N atoms, wherein the carbons of the chain may also be part of a carbonyl group,
R³ and R′³ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R³ and R′³ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms, and
R⁴ and R′⁴ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R⁴ and R′⁴ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms.

The present invention thus concerns bimetallic (Amine)Platinum(II)(NHC) complexes (NHC=N-Heterocyclic Carbene) having trans coordination of the amine and carbene ligands. The two metallic units are connected through the substituent of the N-atom in the NHC ligand.

According to the invention, the expression “Cₜ-C_{z}” means a carbon-based chain which can have from t to z carbon atoms, for example C₁-C₃ means a carbon-based chain which can have from 1 to 3 carbon atoms.

Within the present application, the term “alkyl” means: a linear or branched, saturated, hydrocarbon-based aliphatic group comprising, unless otherwise mentioned, from 1 to 8 carbon atoms. By way of examples, mention may be made of methyl, ethyl, n-propyl, isopropyl, butyl, isobutyl, tert-butyl or pentyl groups.

According to the invention, the term “aryl” means: a cyclic aromatic group comprising between 6 and 10 carbon atoms. By way of examples of aryl groups, mention may be made of phenyl or naphthyl groups.

According to the invention, the term “heteroaryl” means: a 5- to 10-membered aromatic monocyclic or bicyclic group containing from 1 to 4 heteroatoms selected from O, S or N. By way of examples, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl and isoquinolinyl groups.

By way of a heteroaryl comprising 5 to 6 atoms, including 1 to 4 nitrogen atoms, mention may in particular be made of the following representative groups: pyrrolyl, pyrazolyl, 1,2,3-triazolyl, 1,2,4-triazolyl, tetrazolyl and 1,2,3-triazinyl.

Mention may also be made, by way of heteroaryl, of thiophenyl, oxazolyl, furazanyl, 1,2,4-thiadiazolyl, naphthyridinyl, quinoxalinyl, phthalazinyl, imidazo[1,2-a]pyridine, imidazo[2,1 -b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1,2,4-triazinyl, indolizinyl, isoxazolyl, isoquinolinyl, isothiazolyl, purinyl, quinazolinyl, quinolinyl, isoquinolyl, 1,3,4-thiadiazolyl, thiazolyl, isothiazolyl, carbazolyl, and also the corresponding groups resulting from their fusion or from fusion with the phenyl nucleus.

The term “heterocycloalkyl group” means: a 3- to 10-membered, preferably 4- to 10-membered, saturated or partially unsaturated, monocyclic or bicyclic group comprising from one to three heteroatoms selected from O, S or N; the heterocycloalkyl group may be attached to the rest of the molecule via a carbon atom or via a heteroatom; the term bicyclic heterocycloalkyl includes fused bicycles and spiro-type rings.

By way of saturated heterocycloalkyl comprising from 5 to 6 atoms, mention may be made of oxetanyl, tetrahydrofuranyl, dioxolanyl, pyrrolidinyl, azepinyl, oxazepinyl, pyrazolidinyl, imidazolidinyl, tetrahydrothiophenyl, dithiolanyl, thiazolidinyl, tetrahydropyranyl, tetrahydropyridinyl, dioxanyl, morpholinyl, piperidinyl, piperazinyl, tetrahydrothiopyranyl, dithianyl, thiomorpholinyl or isoxazolidinyl.

Among the heterocycloalkyls, mention may also be made, by way of examples, of bicyclic groups such as (8aR)-hexahydropyrrolo[1,2-a]pyrazin-2(1H)-yl, octahydroindozilinyl, diazepanyl, dihydroimidazopyrazinyl and diazabicycloheptanyl groups, or else diazaspiro rings such as 1,7-diazaspiro[4.4]non-7-yl or 1-ethyl-1,7-diazaspiro[4.4]non-7-yl.

When the heterocycloalkyl is substituted, the substitution(s) may be on one (or more) carbon atom(s) and/or on the heteroatom(s). When the heterocycloalkyl comprises several substituents, they may be borne by one and the same atom or different atoms.

The term “cycloalkyl group” means: a cyclic carbon-based group comprising, unless otherwise mentioned, from 3 to 6 carbon atoms. By way of examples, mention may be made of cyclopropyl, cyclobutyl, cyclopentyl, cyclohexyl, etc. groups.

Within the present application, the term “alkanediyl” refers to a divalent aliphatic hydrocarbon radical comprising from 1 to 12 carbon atoms, and preferably from 1 to 6 carbon atoms. Said radical may be represented by the formula (CH₂)ₙ wherein n is an integer varying from 1 to 12, and preferably from 1 to 8.

Within the present application, the term “phenylene” refers to a group divalent group derived from a phenyl group having the formula —C₆H₄—.

Within the present application, the term “heteroarylidene” refers to a group divalent group derived from a heteroaryl group as defined above.

When an alkyl radical is substituted with an aryl group, the term “arylalkyl” or “aralkyl” radical is used. The “arylalkyl” or “aralkyl” radicals are aryl-alkyl-radicals, the aryl and alkyl groups being as defined above. Among the arylalkyl radicals, mention may in particular be made of the benzyl or phenethyl radicals.

The abovementioned “alkyl”, “alkanediyl”, “cycloalkyl”, “aryl”, “phenyl”, “phenylene”, “heteroaryl”, “heteroarylidene” and “heterocycloalkyl” radicals can be substituted with one or more substituents. Among these substituents, mention may be made of the following groups: amino, hydroxyl, thiol, oxo, halogen, alkyl, alkoxy, alkylthio, alkylamino, aryloxy, arylalkoxy, cyano, haloalkyl, trifluoromethyl, carboxyl or carboxyalkyl.

The term “halogen” means: a fluorine, a chlorine, a bromine or an iodine.

The term “alkoxy group” means: an —O-alkyl radical where the alkyl group is as previously defined. By way of examples, mention may be made of —O—(C₁-C₄)alkyl groups, and in particular the —O-methyl group, the —O-ethyl group as —O—C₃alkyl group, the —O-propyl group, the —O-isopropyl group, and as —O—C₄alkyl group, the —O-butyl, —O-isobutyl or —O-tert-butyl group.

The term “alkylthio” means: an —S-alkyl group, the alkyl group being as defined above.

The term “alkylamino” means: an —NH-alkyl group, the alkyl group being as defined above.

The term “aryloxy” means: an —O-aryl group, the aryl group being as defined above.

The term “arylalkoxy” means: an aryl-alkoxy-group, the aryl and alkoxy groups being as defined above.

The term “carboxyalkyl” means: an HOOC-alkyl-group, the alkyl group being as defined above. As examples of carboxyalkyl groups, mention may in particular be made of carboxymethyl or carboxyethyl.

The term “haloalkyl group” means: an alkyl group as defined above, in which one or more of the hydrogen atoms is (are) replaced with a halogen atom. By way of example, mention may be made of fluoroalkyls, in particular CF₃ or CHF₂.

The term “carboxyl” means: a COOH group.

The term “oxo” means: “═O”.

According to an embodiment, in formula (I) as defined above, L is a linker having one of the following formulae:

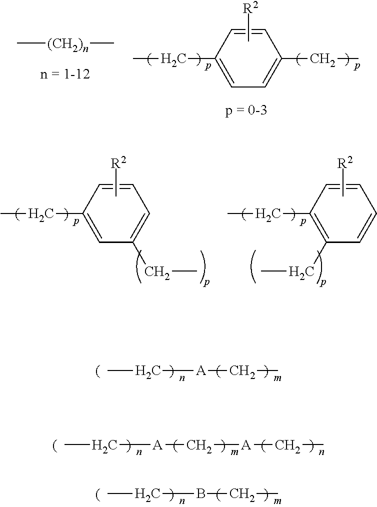

R² being any aryl substituent as defined above,
A being —S—, —O— or —N(R)—, R being for example H or an alkyl group,
m and n being integers comprised between 1 and 10, and
B being a heterocyclic divalent radical.

According to an embodiment, in formula (I) as defined above, L is a C₂-C₁₂ linear chains that may have substituents, such as C₁-C₆ alkyls with linear, branched or cyclic structure, phenyl, substituted phenyl, or heterocyclic radicals.

According to an embodiment, in formula (I) as defined above, L is a phenylene or heteroarylidene group, said groups being possibly substituted as defined above.

According to an embodiment, in formula (I) as defined above, L is an alkanediyl chain that may contain one or more heteroatoms A, with A=O, S, or NR, R being as defined above.

According to a preferred embodiment, the present invention relates to a complex of formula (I) as defined above, wherein L is a C₂-C₁₂ alkanediyl group.

According to a preferred embodiment, in formula (I), R¹ and R² are identical.

According to a preferred embodiment, in formula (I), W¹ and W² are identical.

According to a preferred embodiment, in formula (I), Y¹ and Y² are identical.

According to a preferred embodiment, in formula (I), X¹ and X² are identical.

According to a preferred embodiment, in formula (I), R³ and R′³ are identical.

According to a preferred embodiment, in formula (I), R⁴ and R′⁴ are identical.

Preferably, the complexes of formula (I) are symmetrical compounds.

According to a preferred embodiment, the present invention relates to a complex of formula (I) as defined above, wherein R¹ and R² are C₁-C₆ alkyl groups, preferably methyl.

According to a preferred embodiment, the present invention relates to a complex of formula (I) as defined above, wherein R¹ and R² are C₁-C₆ alkyl groups substituted with a hydroxyl group, and are preferably —(CH₂)₂—OH groups.

According to a preferred embodiment, the present invention relates to a complex of formula (I) as defined above, wherein R¹ and R² are C₁-C₆ alkyl groups substituted with a —C(═O)—NH—(C₆-C₁₀)aryl, and are preferably —CH₂—C(═O)—NH—Ph.

According to a preferred embodiment, the present invention relates to a complex of formula (I) as defined above, wherein R³ or R′³ and R⁴ or R′⁴ are H or a cycloalkyl group, preferably H or a cyclohexyl group.

According to a preferred embodiment, the present invention relates to a complex of formula (I) as defined above, wherein R³ or R′³ and R⁴ or R′⁴ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms.

According to a preferred embodiment, in formula (I) as defined above, Y¹, Y², W¹ and W² are a CH group.

A preferred group of complexes according to the invention consists of complexes having the following formula (II):

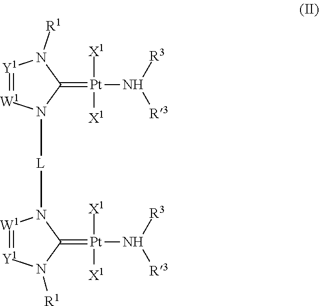

R¹, X¹, Y¹, W¹, L, R³, and R′³ being as defined above in formula (I).

Another preferred group of complexes according to the invention consists of complexes having the following formula (III):

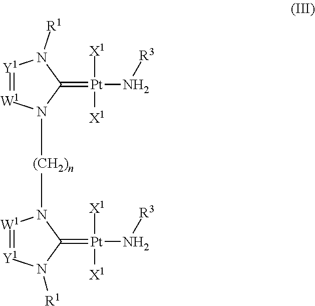

R¹, X¹, Y¹, W¹, and R³ being as defined above in formula (I), and
n being an integer comprised between 1 and 12, and being preferably 2, 4, 6, or 8.

Another preferred group of complexes according to the invention consists of complexes having the following formula (IV):

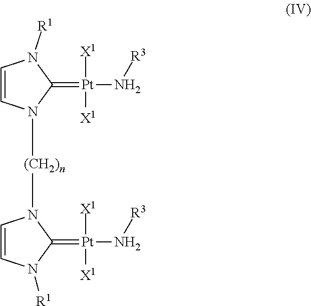

X¹, R¹, and R³ being as defined above in formula (I), and
n being an integer comprised between 1 and 12, and being preferably 2, 4, 6, or 8.

Another preferred group of complexes according to the invention consists of complexes having the following formula (V):

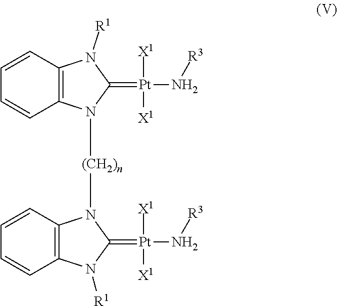

X¹, R¹, and R³ being as defined above in formula (I), and
n being an integer comprised between 1 and 12, and being preferably 2, 4, 6, or 8.

Preferably, in formula (II), (III), (IV) or (V), X¹ is iodide.

Preferably, in formula (II), (III), (IV) or (V), R¹ is an alkyl group, such as methyl.

The present invention also relates to the preferred complexes having one of the following formulae:

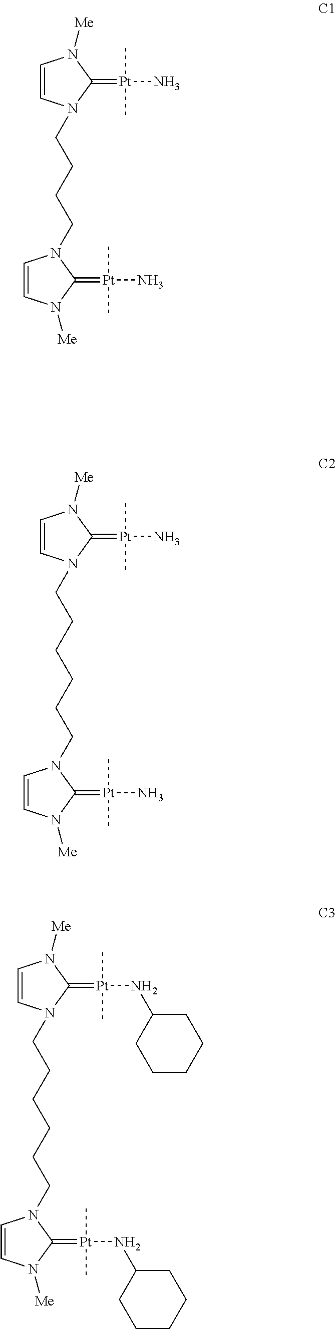

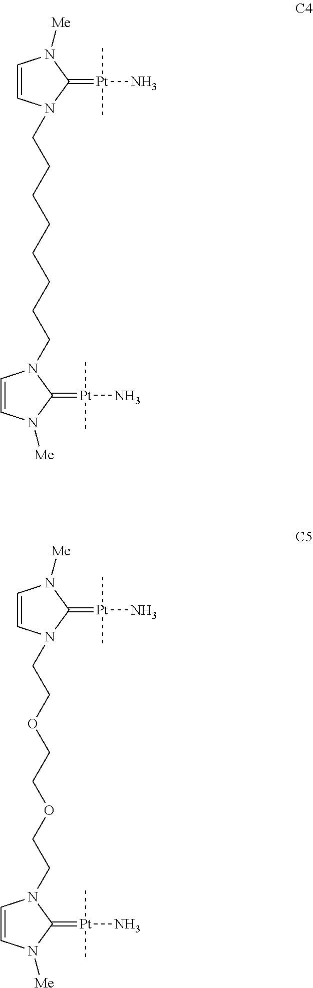

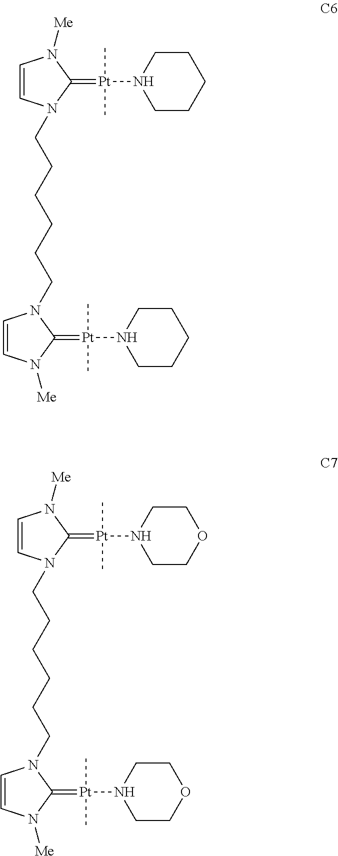

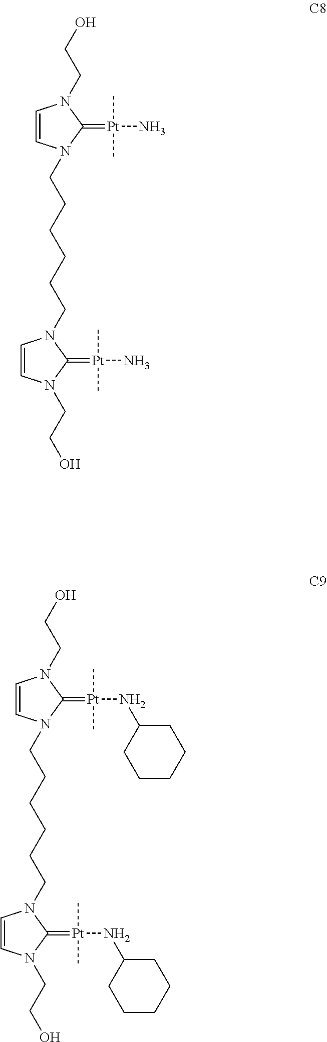

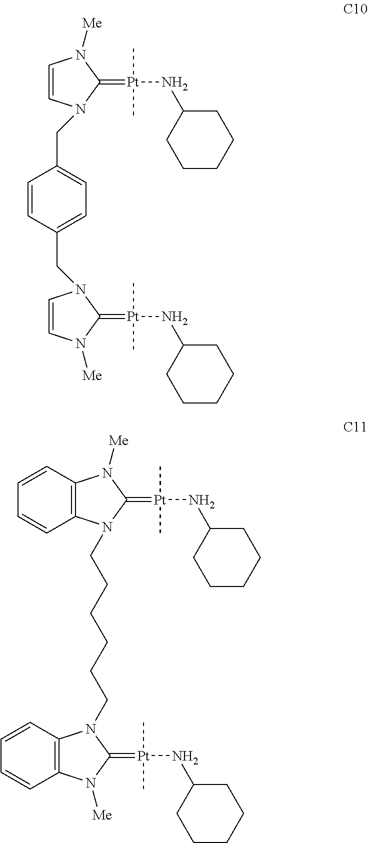

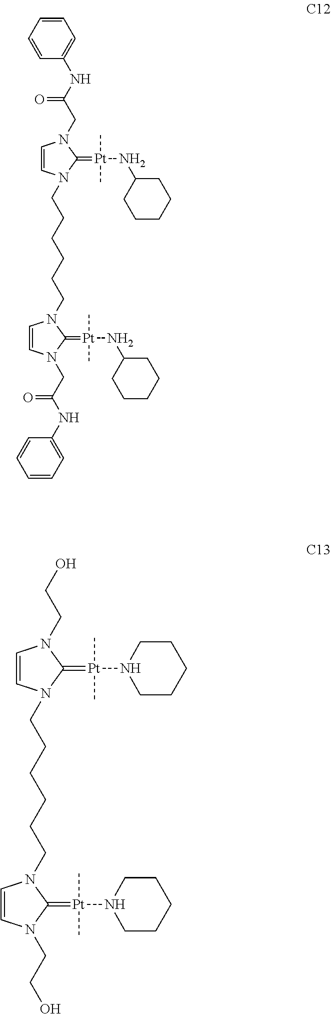

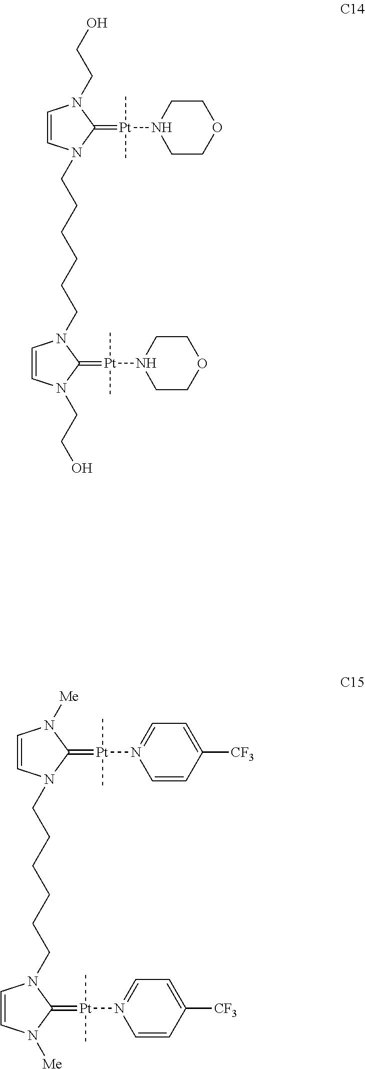

The present invention also relates to a complex having one of the following formulae:

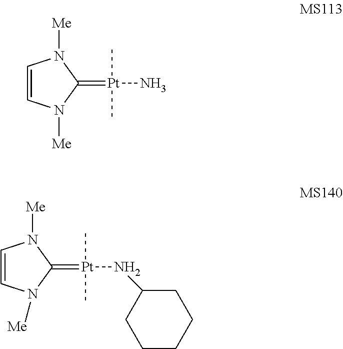

The present invention also relates to a conjugate molecule comprising one or more complexe(s) according to the invention, in particular those having the formula (I) as defined above, said complexe(s) being covalently bound to at least one cell binding agent.

The present invention also relates to a conjugate molecule comprising one or more complexe (s) according to the invention, in particular those having the formula (II), (III), (IV) or (V) as defined above, said compound(s) being covalently bound to at least one cell binding agent.

The present invention also relates to a conjugate molecule being a complex according to the invention to which is bound to at least one payload, optionally through a linker or covalently conjugated, thereby forming a single molecule.

Examples of suitable payloads include, but are not limited to, peptides, polypeptides, proteins, antibodies, or antigen-binding fragments thereof, antigens, nucleic acid molecules, polymers, small molecules, mimetic agents, drugs, inorganic molecules, organic molecules, and radioisotopes.

Examples of suitable payloads include, but are not limited to, cell binding agents, chemotherapeutic agents, targeted therapy agents, cytotoxic agents, ligands for cellular receptor(s), immunomodulatory agents, pro-apoptotic agents, anti-angiogenic agents, photodetectable labels, contrast agents, radiolabels, and the like.

According to the invention, a cell binding agent is a molecule with affinity for a biological target. The cell binding agent may be, for example, a ligand, a protein, an antibody, more particularly a monoclonal antibody, a protein or antibody fragment, a peptide, an oligonucleotide or an oligosaccharide. The function of the binding agent is to direct the biologically active compound towards the biological target.

In some embodiments, the conjugate of the invention is an antibody drug conjugate (ADC). Accordingly, the payload is an antibody, particularly monoclonal antibody, sdAb, VHH, intrabody, or single-chain antibodies (scFv).

In some embodiments, the conjugate of the invention comprises a linker unit between the complex of the invention and the payload. In some embodiments, the linker is cleavable under intracellular conditions, such that cleavage of the linker releases the complex of the invention from the payload in the intracellular environment. In yet other embodiments, the linker unit is not cleavable and the complex of the invention is released, for example, by payload degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease.

In some embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661). Such linkers are relatively stable under neutral pH conditions, such as those in the blood, but are unstable at below pH 5.5 or 5.0, the approximate pH of the lysosome.

In some embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935).

The present invention also relates to the complex according to the invention, in particular one having the formula (I) as defined above or the conjugate as mentioned above, for use as drug.

The present invention also relates to the complex according to the invention, in particular one having the formula (II), (III), (IV) or (V) as defined above for use as drug.

The present invention also relates to a medicament comprising a complex according to the invention, in particular one of formula (I) as defined above, or the conjugate as defined above, or a pharmaceutically acceptable salt thereof.

The present invention also relates to a medicament comprising a complex according to the invention, in particular one of formula (II), (III), (IV) or (V) as defined above, or a pharmaceutically acceptable salt thereof.

The present invention also relates to a pharmaceutical composition, comprising a complex according to the invention, in particular one of formula (I) as defined above or the conjugate as defined above, or a pharmaceutically acceptable salt thereof, and also at least one pharmaceutically acceptable excipient.

The present invention also relates to a pharmaceutical composition, comprising a complex of formula (II), (III), (IV) or (V) as defined above, or a pharmaceutically acceptable salt thereof, and also at least one pharmaceutically acceptable excipient.

These pharmaceutical compositions contain an effective dose of at least one complex according to the invention, or a pharmaceutically acceptable salt, and also at least one pharmaceutically acceptable excipient.

Said excipients are selected, according to the pharmaceutical form and the mode of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or the salt thereof, can be administered in unit administration form, as a mixture with conventional pharmaceutical excipients, to animals and to human beings for the treatment of the disorders and diseases below.

The suitable unit administration forms include oral forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

The present invention also relates to a complex according to the invention, in particular one of formula (I) as defined above or the conjugate as defined above, for use in treating cancer.

The present invention also relates to a complex of formula (II), (III), (IV) or (V) as defined above for use in treating cancer.

The present invention also relates to a complex according to the invention, in particular one of formula (I) as defined above or the conjugate as defined above, for use in treating cancer in combination with radiotherapy and/or in combination with radiotherapy and any anticancer drug.

The present invention also relates to a compound of formula (II), (III), (IV) or (V) as defined above, for use in treating cancer in combination with radiotherapy and/or in combination with radiotherapy and any anticancer drug.

In some embodiments, the complex, the conjugate and/or the pharmaceutical composition according to the invention is administered in combination with additional cancer therapies. In particular, the complex, the conjugate and/or the pharmaceutical composition of the invention may be administered in combination without or with targeted therapy, immunotherapy such as immune checkpoint therapy and immune checkpoint inhibitor, co-stimulatory antibodies, or chemotherapy.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Co-stimulatory antibodies deliver positive signals through immune-regulatory receptors including but not limited to ICOS, CD137, CD27, OX-40 and GITR.

Examples of anti-PD1 antibodies include, but are not limited to, nivolumab, cemiplimab (REGN2810 or REGN-2810), tislelizumab (BGB-A317), tislelizumab, spartalizumab (PDR⁰⁰¹ or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in international applications WO2004004771, WO2004056875, WO2006121168, WO2008156712, WO2009014708, WO2009114335, WO2013043569 and WO2014047350.

Examples of anti-PD-L1 antibodies include, but are not limited to, LY3300054, atezolizumab, durvalumab and avelumab.

Examples of anti-CTLA-4 antibodies include, but are not limited to, ipilimumab (see, e.g., U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., international applications WO1997020574 and WO2007123737) and antibodies described in U.S. Pat. No. 8,491,895.

Examples of anti-VISTA antibodies are described in US patent application US20130177557.

Examples of inhibitors of the LAG3 receptor are described in U.S. Pat. No. 5,773,578.

Example of KIR inhibitor is IPH4102 targeting KIR³DL2.

In some embodiments, the complex, the conjugate and/or the pharmaceutical composition of the invention is administered to the patient in combination with targeted therapy. Targeted therapy agents, are drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF),(Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the complex, the conjugate and/or the pharmaceutical composition of the invention is administered to the patient in combination with chemotherapy. As used herein, the term “chemotherapy” has its general meaning in the art and refers to the treatment that consists in administering to the patient a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

According to an embodiment, the compound, the conjugate and/or the pharmaceutical composition of the invention is administered to the patient in combination with radiotherapy. The present invention thus concerns new radiosensitizers, preferably used in combination with radiation.

Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, fractionated stereotactic radiation therapy, hypofractionated radiotherapy, cobalt therapy, electron therapy, fast neutron therapy, neutron-capture therapy, proton therapy, intensity modulated radiation therapy (IMRT), 3-dimensional conformal radiation therapy (3D-CRT) and the like; brachytherapy; unsealed source radiotherapy; tomotherapy and the like; or minibeam radiation therapy. Gamma rays are another form of photons used in radiotherapy. Gamma rays are produced spontaneously as certain elements (such as radium, uranium, cesium and cobalt 60) release radiation as they decompose, or decay. In some embodiments, radiotherapy may be hadrontherapy (using beams from charged particles like protons or other ions such as carbon), proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11). Radiotherapy may also be hypofractionated radiotherapy (Shah J L, Li G, Shaffer J L, Azoulay M I, Gibbs I C, Nagpal S, Soltys S G. Stereotactic Radiosurgery and Hypofractionated Radiotherapy for Glioblastoma. Neurosurgery. 2018 Jan. 1; 82(1):24-34, doi: 10.1093/neuros/nyx115; Botti M, Kirova Y M, Dendale R, Savignoni A, Fromantin I, Gautier C, Bollet M A, Campana F, Fourquet A. Hypofractionated breast radiotherapy in 13 fractions, perfect tolerance or delayed early reaction? Prospective study of Curie Institute. Cancer Radiother. 2009 April; 13(2):92-6, doi: 10.1016/j.canrad.2008.11.009).

According to an embodiment, the cancer is selected from the group consisting of: glioblastoma, lung cancer, non-small cell lung cancer (NSCLC), ovarian cancer, bladder cancer, rectal cancer, cervical cancer, and head and neck cancer.

The compounds or conjugates according to the invention are particularly deemed useful for the treatment of cancer including solid tumors such as skin, breast, brain, cervical carcinomas, testicular carcinomas rectum carcinoma, anal carcinoma, etc. More particularly, cancers that may be treated by the compounds or conjugates of the invention include, but are not limited to: cardiac sarcoma (angiosarcoma, fibrosarcoma, rhabdomyosarcoma, liposarcoma), myxoma, rhabdomyoma, fibroma, lipoma and teratoma; lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma, gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, Villous adenoma, hamartoma, leiomyoma); Genitourinary tract: kidney (adenocarcinoma, Wilm's tumor nephroblastoma, lymphoma, leukemia), bladder and urethra (Squamous cell carcinoma, transitional cell carcinoma, adenocarcinoma), prostate (adenocarcinoma, Sarcoma), testis (seminoma, teratoma, embryonal carcinoma, teratocarcinoma, choriocarcinoma, Sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell Sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors, Nervous System: skull (osteoma, hemangioma, granuloma, Xanthoma, Osteitis deformans), meninges (meningioma, meningiosarcoma, gliomatosis), brain (astrocytoma, medulloblastoma, glioma, ependymoma, germinomapinealoma, glioblastoma multiform, oligodendroglioma, Schwannoma, retinoblastoma, congenital tumors), Spinal cord (neurofibroma, meningioma, glioma, Sarcoma); Gynecological: uterus (endometrial carcinoma), cervix (cervical carcinoma, pre-tumor cervical dysplasia), ovaries (ovarian carcinoma, Serous cystadenocarcinoma, mucinous cystadenocarcinoma, unclassified carcinoma, granulosa-thecal cell tumors, Sertoli-Leydig cell tumors, dysgerminoma, malignant teratoma), Vulva (Squamous cell carcinoma, intraepithelial carcinoma, adenocarcinoma, fibrosarcoma, melanoma), vagina (clear cell carcinoma, Squamous cell carcinoma, botryoid Sarcoma embryonal rhabdomyosarcoma, fallopian tubes (carcinoma); Hematologic: blood (myeloid leukemia acute and chronic, acute lymphoblastic leukemia, chronic lymphocytic leukemia, myeloproliferative diseases, multiple myeloma, myelodysplastic Syndrome), Hodgkin's disease, non-Hodgkin's lymphoma malignant lymphoma; Skin: malignant melanoma, basal cell carcinoma, Squamous cell carcinoma, Karposi's Sarcoma, moles dysplastic nevi, lipoma, angioma, dermatofibroma, keloids, psoriasis, and Adrenal glands: neuroblastoma.

According to an embodiment, the cancer is selected from the group consisting of: benign, metastatic and malignant neoplasias, and also including acral lentiginous melanoma, actinic keratoses, adenocarcinoma, adenoid cycstic carcinoma, adenomas, adenosarcoma, adenosquamous carcinoma, astrocytic tumors, Bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma. neuroepithelial adenocarcinoma nodular melanoma, oat cell carcinoma, oligodendroglial, osteosarcoma, pancreatic polypeptide. papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma. and Wilm's tumor.

The present invention also relates to a method for treating the pathological conditions indicated above, which comprises the administration, to a patient, of an effective dose of a complex according to the invention, or a pharmaceutically acceptable salt thereof.

### FIGURES

FIG. 1: Survival curves of A2780 (A) and H1299 cells lines (B) following irradiation in the absence or in the presence of MS140 or complex C2. Comparison of the D10 values of the complexes evaluated at 1 μM in A2780 (C) and HT1299 (D) cell lines.

FIG. 2: Ratio of the D10 values of monometallic complexes MS140 (A and C) and MS113 (B and D) in A2780 (A and B) and H1299 (C and D) cell lines as function of their concentration.

FIG. 3: Ratio of the D10 values of bimetallic complexes C1, C2, C3 and C4 in A2780 cell lines (A, B, C, D) and C2 and C4 in H1299 cell lines (E, F) as function of their concentration.

FIG. 4: Ratio of the D10 values of bimetallic complex C2 in IC5, CRL1550 and CC11⁺ as function of their concentration.

FIG. 5: DNA damage repair kinetics. Number of γ-H2AX foci in A2780 cells after irradiation at 2 Gy. The cells were previously not pre-treated or pre-treated by MS113, MS140 or C2

### EXAMPLES

### Preparation of Complexes According to the Invention

### [1,1′-(Butane-1,4-diyl)bis(3-methylimidazol-2-ylidene)]bis[(1,3-divinyl-1,3,3-tetramethyldisiloxane)platinum]

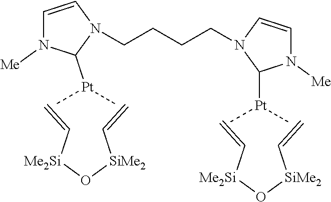

To a suspension of 1,4-bis(3-methylimidazolium-1-yl)butane dibromide (Nachtigall, F. M.; Corilo, Y. E.; Cassol, C. C.; Ebeling, G.; Morgon, N. H.; Dupont, J.; Eberlin, M. N. Angew. Chem. Int. Ed. 2008, 47, 151-154) (380 mg, 1.00 mmol, 1 equiv.) in dichloromethane (5 mL) under argon was added the Platinum⁽⁰⁾-1,3-divinyl-1,1,3,3-tetramethyldisiloxane complex (solution in xylene, ˜2% Platinum concentration, 23.0 mL, 2.00 mmol, 2 equiv.). The mixture was cooled to 0° C., t-BuOK (315 mg, 2.80 mmol, 2.8 equiv.) was added in one portion and the mixture was slowly allowed to warm to r.t. and stirred for 24 hours. Then silica gel was added and the solvents were removed under reduced pressure to provide a solid sample loading for column chromatography. The crude product was purified over silica gel (eluent: n-heptane/EtOAc 7:3) to afford the title compound as a colorless oil (400 mg, 41% yield).

¹H NMR (CDCl₃, 300.2 MHz) δ 6.98 (s, 2H, H_{Imi}), 6.92 (s, 2H, H_{Imi}), 3.83 (bs, 4H, NCH₂), 3.49 (s, 6H, NCH₃), 2.31-2.05 (m, 4H, C═CH₂), 1.92-1.72 (m, 8H, C═CH₂, SiCH), 1.60 (bs, 4H, CH₂), 0.33 (s, 12H, SiCH₃), −0.29 (s, 12H, SiCH₃).

¹³C NMR (CDCl₃, 75.5 MHZ) δ 183.7 (Pt-C_{car}), 122.2 (J ¹³C-¹⁹⁵Pt=37.7 Hz, C_{Imi}), 120.4 (J ¹³C-¹⁹⁵Pt=36.7 Hz, C_{Imi}), 49.4 (J ¹³C-¹⁹⁵Pt=41.0 Hz, NCH₂), 40.2 (J ¹³C-¹⁹⁵PT=157.7 Hz, SiCH═CH₂), 37.0 (J ¹³C-¹⁹⁵Pt=45.2 Hz, NCH₃), 34.3 (J ¹³C-¹⁹⁵Pt=118.1 Hz, SiCH═CH₂), 27.6 (CH₂), 1.6 (SiCH₃), −1.6 (SiCH₃).

### [1,1′-(Hexane-1,6-diyl)bis(3-methylimidazol-2-ylidene)]bis[(1,3-divinyl-1,1,3,3-tetramethyldisiloxane)platinum]

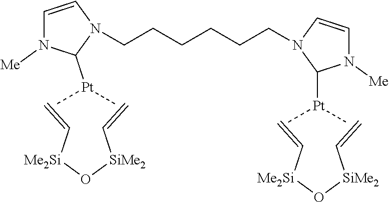

To a suspension of 1,6-bis(3-methylimidazolium-1-yl)hexane dibromide (Anderson, J. L.; Ding, R.; Ellern, A.; Armstrong, D. W. J. Am. Chem. Soc. 2005, 127, 593-604) (391 mg, 0.96 mmol, 1 equiv.) in dichloromethane (6 mL) under argon was added the Platinum⁽⁰⁾-1,3-divinyl-1,1,3,3-tetramethyldisiloxane complex (solution in xylene, ˜2% Platinum concentration, 22.0 mL, 1.92 mmol, 2 equiv.). The mixture was cooled to 0° C., t-BuOK (301 mg, 2.68 mmol, 2.8 equiv.) was added in one portion and the mixture was slowly allowed to warm to r.t. and stirred for 48 hours. Then silica gel was added and the solvents were removed under reduced pressure to provide a solid sample loading for column chromatography. The crude product was purified over silica gel (eluent: n-heptane/EtOAc 7:3) to afford the title compound as a colorless oil (889 mg, 92% yield).

¹H NMR (CDCl₃, 500.2 MHz) δ 6.98 (s, 2H, H_{Imi}), 6.96 (s, 2H, H_{Imi}), 3.82 (t, J=7.2 Hz, 4H, NCH₂), 3.50 (s, 6H, NCH₃), 2.26-2.12 (m, 4H, C═CH₂), 1.93-1.75 (m, 8H, C═CH₂, SiCH), 1.61 (t, J=7.2 Hz, 4H, CH₂), 1.19 (t, J=7.2 Hz, 4H, CH₂), 0.33 (s, 12H, SiCH₃), −0.29 (s, 12H, SiCH₃).

¹³C NMR (CDCl₃, 75.5 MHZ) δ 183.5 (Pt-C_{car}), 122.0 (J ¹³C-¹⁹⁵Pt=36.4 Hz, C_{Imi}), 120.4 (J ¹³C-¹⁹⁵Pt=37.1 Hz, C_{Imi}), 49.8 (J ¹³C-¹⁹⁵Pt=40.6 Hz, NCH₂), 40.1 (J ¹³C-¹⁹⁵Pt=159.2 Hz, SiCH═CH₂), 37.0 (J ¹³C-¹⁹⁵Pt=44.3 Hz, NCH₃), 34.1 (J ¹³C-¹⁹⁵Pt=119.1 Hz, SiCH═CH₂), 30.5 (CH₂), 26.3 (CH₂), 1.6 (SiCH₃), −1.7 (SiCH₃).

IR (v/cm⁻¹) 2952, 1456, 1401, 1296, 1244, 1170, 986, 859, 835, 780, 678.

HRMS (ESI+) calcd. for C₃₀H₅₈N₄O₂Si₄Pt₂Na [M+Na]⁺: 1031.2830, found: 1031.2823.

### [1,1′-(Octane-1,8-diyl)bis[(3-methylimidazol-2-ylidene)]bis[(1,3-divinyl-1,1,3,3-tetramethyldisiloxane)platinum]

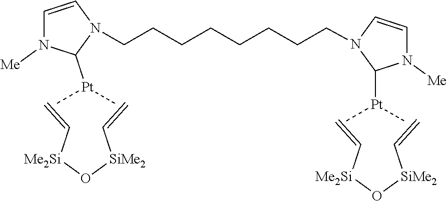

To a suspension of 1,8-bis(3-methylimidazolium-1-yl)octane dibromide (Gindri, I. M.; Siddiqui, D. A.; Bhardwaj, P.; Rodriguez, L. C.; Palmer, K. L.; Frizzo, C. P.; Martins, M. A. P.; Rodrigues, D. C. RSC Advances 2014, 4, 62594-62602) (417 mg, 0.96 mmol, 1 equiv.) in dichloromethane (6 mL) under argon was added the Platinum⁽⁰⁾-1,3-divinyl-1,1,3,3-tetramethyldisiloxane complex (solution in xylene, ˜2% Platinum concentration, 22.0 mL, 1.92 mmol, 2 equiv.). The mixture was cooled to 0° C., t-BuOK (300 mg, 2.68 mmol, 2.8 equiv.) was added in one portion and the mixture was slowly allowed to warm to r.t. and stirred for 48 hours. Then silica gel was added and the solvents were removed under reduced pressure to provide a solid sample loading for column chromatography. The crude product was purified over silica gel (eluent: n-heptane/EtOAc 7:3) to afford the title compound as a colorless oil (871 mg, 88% yield).

¹H NMR (CDCl₃, 500.2 MHz) δ 6.99 (bs, 4H, H_{Imi}), 3.84 (t, J=7.7 Hz, 4H, NCH₂), 3.50 (s, 6H, NCH₃), 2.26-2.13 (m, 4H, C═CH₂), 1.94-1.76 (m, 8H, C═CH₂, SiCH), 1.63 (bs, 4H, CH₂), 1.19 (bs, 8H, CH₂), 0.32 (s, 12H, SiCH₃), −0.29 (s, 12H, SiCH₃).

¹³C NMR (CDCl₃, 75.5 MHZ) δ 183.4 (Pt-C_{car}), 122.0 (J ¹³C-¹⁹⁵Pt=36.4 Hz, C_{Imi}), 120.4 (J ¹³C-¹⁹⁵Pt=38.4 Hz, C_{Imi}), 50.0 (J ¹³C-¹⁹⁵Pt=39.4 Hz, NCH₂), 40.1 (J ¹³C-¹⁹⁵Pt=158.1 Hz, SiCH═CH₂), 36.9 (J ¹³C-¹⁹⁵Pt=45.6 Hz, NCH₃), 34.1 (J ¹³C-¹⁹⁵Pt=118.2 Hz, SiCH═CH₂), 30.6 (CH₂), 29.1 (CH₂), 26.6 (CH₂), 1.6 (SiCH₃), −1.7 (SiCH₃).

IR (v/cm⁻¹) 2930, 2857, 1456, 1403, 1296, 1245, 1170, 1002, 989, 859, 835, 780, 678.

HRMS (ESI+) calcd. for C₃₂H₆₂N₄O₂Si₄Pt₂Na [M+Na]⁺: 1059.3143, found: 1059.3125.

### [1,1′-(3,5-Dioxa-octane-1,8-diyl)bis(3-methylimidazol-2-ylidene)]bis[(1,3-divinyl-1,1,3,3-tetramethyldisiloxane)platinum]

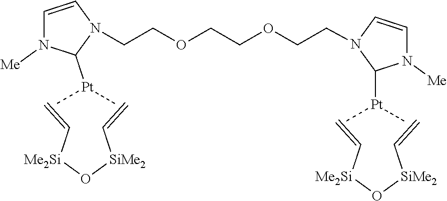

To a solution of 1,6-bis(3-methylimidazolium-1-yl)-3,5-dioxa-octane dibromide (Kowsari, E.; Abdpour, S. J. Solid State Chem. 2017, 256, 141-150) (88 mg, 0.25 mmol, 1 equiv.) in dichloromethane (2 mL) under argon was added the Platinum⁽⁰⁾-1,3-divinyl-1,1,3,3-tetramethyldisiloxane complex (solution in xylene, ˜2% Platinum concentration, 5.6 mL, 0.49 mmol, 2.0 equiv.). The mixture was cooled to 0° C., t-BuOK (56 mg, 0.50 mmol, 2.0 equiv.) was added in one portion. The mixture was heated to 35° C. for 1 h and stirred at r.t. for 24 h. Then silica gel was added and the solvents were removed under reduced pressure to provide a solid sample loading for column chromatography. The crude product was purified over silica gel (eluent: n-heptane/EtOAc 7:3 to 6:4) to afford the title compound as a pale yellow solid (178 mg, 68% yield).

¹H NMR (CDCl₃, 300.2 MHz) δ 7.15 (d, J=2.0 Hz, J ¹H-¹⁹⁵Pt=12 Hz, 2H, H_{Imi}), 6.95 (d, J=2.0 Hz, J ¹H-¹⁹⁵Pt=12 Hz, 2H, H_{Imi}), 4.07 (t, J=5.5 Hz, 4H, CH₂), 3.60 (t, J=5.5 Hz, 4H, CH₂), 3.53 (bs, 4H, CH₂), 3.50 (bs, 6H, NCH₃), 2.19 (d, J=10.0 Hz, J ¹H-¹⁹⁵Pt=53 Hz, 4H, C═CH₂), 1.97-1.68 (m, 8H, C═CH₂, SiCH), 0.32 (s, 12H, SiCH₃), −0.29 (s, 12H, SiCH₃).

¹³C NMR (CDCl₃, 75.5 MHZ) δ 183.8 (Pt-C_{car}), 122.2 (J ¹³C-¹⁹⁵Pt=38 Hz, C_{Imi}), 121.7 (J ¹³C-¹⁹⁵Pt=37 Hz, C_{Imi}), 70.7 (OCH₂), 70.6 (OCH₂), 49.8 (J ¹³C-¹⁹⁵Pt=40 Hz, NCH₂), 40.3 (J ¹³C-¹⁹⁵Pt=157 Hz, SiCH═CH₂), 36.9 (J ¹³C-¹⁹⁵Pt=45 Hz, NCH₃), 34.4 (J ¹³C-¹⁹⁵Pt=118 Hz, SiCH═CH₂), 1.6 (SiCH₃), −1.7 (SiCH₃).

IR (v/cm⁻¹) 3009, 2953, 2896, 1452, 1400, 1295, 1244, 1203, 1169, 1111, 1002, 987, 859, 835, 779, 732, 706.

HRMS (ESI+) calcd. for C₃₀H₅₈N₄O₄Si₄Pt₂Na [M+Na]⁺: 1063.2729, found: 1063.2676.

### {1,1′-(Hexane-1,6-diyl)bis[3-(2-hydroxethyl)imidazol-2-ylidene]}bis[(1,3-divinyl-1,1,3,3-tetramethyldisiloxane)platinum]

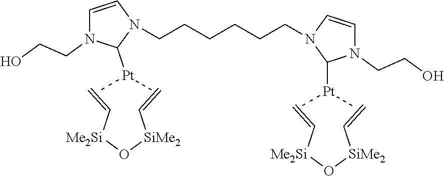

To a solution of 1,6-bis(3-(2-hydroxethyl)imidazolium-1-yl)hexane dibromide (500 mg, 1.07 mmol, 1 equiv.) in ethanol (20 mL) under argon was added the Platinum⁽⁰⁾-1,3-divinyl-1,1,3,3-tetramethyldisiloxane complex (solution in xylene, ˜2% Platinum concentration, 26 mL, 2.35 mmol, 2.2 equiv.). The mixture was cooled to 0° C., t-BuOK (671 mg, 5.98 mmol, 5.6 equiv.) was added in one portion and the mixture was slowly allowed to warm to r.t. and stirred for 5 hours. Then silica gel was added and the solvents were removed under reduced pressure to provide a solid sample loading for column chromatography. The crude product was purified over silica gel (eluent: n-heptane/EtOAc 5:5) to afford the title compound as a colorless oil (444 mg, 39% yield).

¹H NMR (CDCl₃, 500.2 MHz) δ 7.15 (s, 2H, H_{Imi}), 6.99 (s, 2H, H_{Imi}), 4.06 (bs, 4H, CH₂N), 3.84-3.79 (m, 8H, NCH₂, HOCH₂), 2.26-2.13 (m, 4H, C═CH₂), 1.90-1.77 (m, 10H, C═CH₂, SiCH, OH), 1.61 (bs, 4H, CH₂), 1.19 (bs, 4H, CH₂), 0.32 (s, 12H, SiCH₃), −0.30 (s, 12H, SiCH₃).

¹³C NMR (CDCl₃, 125.8 MHz) δ 182.7 (Pt-C_{car}), 121.9 (J ¹³C-¹⁹⁵Pt=37 Hz, C_{Imi}), 120.4 (C_{Imi}), 62.3 (HOCH₂), 52.3 (NCH₂), 50.0 (CH₂N), 41.0 (J ¹³C-¹⁹⁵Pt=160 Hz, SiCH═CH₂), 34.6 (J ¹³C-¹⁹⁵Pt=118 Hz, SiCH═CH₂), 30.5 (CH₂), 26.2 (CH₂), 1.4 (SiCH₃), −1.8 (SiCH₃).

HRMS (ESI+) calcd. for C₃₂H₆₂N₄O₄Si₄Pt₂Na [M+Na]⁺: 1091.3041, found: 1091.3031.

### [1,1′-(Benzene-1,4-bis-methylenediyl)bis(3-methylimidazol-2-ylidene)]bis[(1,3-divinyl-1,1,3,3-tetramethyldisiloxane)platinum]

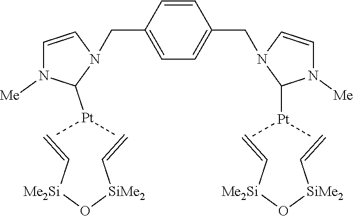

To a solution of 1,4-bis-methylene(3-methylimidazolium-1-yl)-benzene dibromide (Jiao, D.; Biedermann, F.; Scherman, O. A. Org. Lett. 2011, 13, 3044-3047) (321 mg, 0.75 mmol, 1 equiv.) in dichloromethane (15 mL) under argon was added the Platinum⁽⁰⁾-1,3-divinyl-1,1,3,3-tetramethyldisiloxane complex (solution in xylene, ˜2% Platinum concentration, 17 mL, 1.51 mmol, 2.0 equiv.). The mixture was cooled to 0° C., t-BuOK (168 mg, 1.51 mmol, 2.0 equiv.) was added in one portion and the mixture was slowly allowed to warm to r.t. and stirred for 48 hours. Then silica gel was added and the solvents were removed under reduced pressure to provide a solid sample loading for column chromatography. The crude product was purified over silica gel (eluent: n-heptane/EtOAc 7:3 to 6:4) to afford the title compound as a colorless solid (563 mg, 73% yield).

¹H NMR (CDCl₃, 300.2 MHz) δ 7.05 (bs, 4H, H), 7.02 (d, J=1.9 Hz, J ¹H-¹⁹⁵Pt=12 Hz, 2H, H_{Imi}), 6.87 (d, J=1.9 Hz, J ¹H-¹⁹⁵Pt=12 Hz, 2H, H_{Imi}), 5.10 (s, 4H, CH₂), 3.55 (bs, 6H, NCH₃), 2.29-2.10 (bs, 4H, C═CH₂), 1.97-1.74 (m, 8H, C═CH₂, SiCH), 0.30 (s, 12H, SiCH₃), −0.36 (bs, 12H, SiCH₃).

¹³C NMR (CDCl₃, 75.5 MHZ) δ 185.0 (Pt-C_{car}), 127.9 (CH), 122.7 (J ¹³C-¹⁹⁵Pt=37 Hz, C_{Imi}), 120.7 (J ¹³C-¹⁹⁵Pt=37 Hz, C_{Imi}), 53.1 (J ¹³C-¹⁹⁵Pt=42 Hz, NCH₂), 40.5 (J ¹³C-¹⁹⁵Pt=157 Hz, SiCH═CH₂), 37.0 (J ¹³C-¹⁹⁵Pt=46 Hz, NCH₃), 34.7 (J ¹³C-¹⁹⁵Pt=118 Hz, SiCH═CH₂), 1.6 (SiCH₃), −1.8 (SiCH₃).

IR (v/cm⁻¹) 2954, 1452, 1395, 1297, 1244, 1171, 984, 908, 836, 779, 732, 707.

HRMS (ESI+) calcd. for C₃₂H₅₅N₄O₂Si₄Pt₂ [M+H]⁺: 1029.2698, found: 1029.2732.

### [1,1′-(Hexane-1,6-diyl)bis(3-methylbenzo-imidazol-2-ylidene)]bis[(1,3-divinyl-1,1,3,3-tetramethyldisiloxane) platinum]

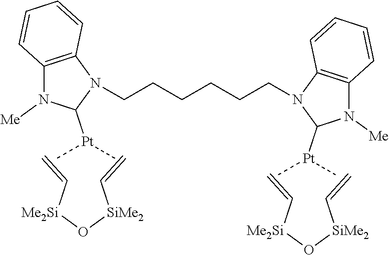

To a solution of 1,6-bis(3-methylbenzo-imidazolium-1-yl)hexane dibromide (Mezzetta, A.; Guglielmero, L.; Mero, A.; Tofani, G.; D'Andrea, F.; Pomelli, C. S.; Guazzelli, L. Molecules 2021, 26, 4211) (320 mg, 0.63 mmol, 1 equiv.) in dichloromethane (10 mL) under argon was added the Platinum⁽⁰⁾-1,3-divinyl-1,1,3,3-tetramethyldisiloxane complex (solution in xylene, ˜2% Platinum concentration, 14 mL, 1.26 mmol, 2.0 equiv.). The mixture was cooled to 0° C., t-BuOK (141 mg, 1.26 mmol, 2.0 equiv.) was added in one portion and the mixture was slowly allowed to warm to r.t. and stirred for 16 hours. Then silica gel was added and the solvents were removed under reduced pressure to provide a solid sample loading for column chromatography. The crude product was purified over silica gel (eluent: n-heptane/EtOAc 95:5 to 8:2) to afford the title compound as a colorless solid (500 mg, 72% yield).

¹H NMR (CDCl₃, 500.2 MHz) δ 7.36-7.26 (m, 8H), 4.14 (bs, 4H, CH₂), 3.71 (s, 6H, NCH₃), 2.33-2.18 (bs, 4H, C═CH₂), 1.95-1.79 (m, 8H, C═CH₂, SiCH), 1.73 (bs, 4H, CH₂), 1.27 (bs, 4H, CH₂), 0.36 (s, 12H, SiCH₃), −0.27 (bs, 12H, SiCH₃).

### {1,1-(Hexane-1,6-diyl)bis[3-(2-oxo-2-(phenylamino)ethyl)imidazol-2-ylidene]}bis[(1,3-divinyl-1,1,3,3-tetramethyldisiloxane)platinum]

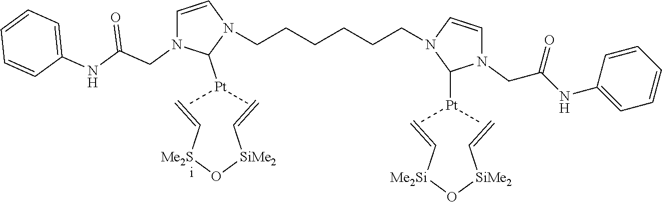

To a solution of 1,6-bis(3-(2-oxo-2-(phenylamino)ethyl)imidazolium-1-yl)hexane dibromide (500 mg, 0.67 mmol, 1 equiv.) in acetone (20 mL) under argon was added the Platinum⁽⁰⁾-1,3-divinyl-1,1,3,3-tetramethyldisiloxane complex (solution in xylene, ˜2% Platinum concentration, 16 mL, 1.35 mmol, 2.0 equiv.). The mixture was cooled to 0° C., K₂CO₃ (933 mg, 6.75 mmol, 10 equiv.) was added in one portion and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 7:3) to afford the title compound as a colorless oil (130 mg, 15% yield).

¹H NMR (CDCl₃, 300.2 MHz) δ 7.68 (bs, 2H, NH), 7.37-7.29 (m, 8H, H), 7.22 (bs, 2H, H_{Imi}), 7.13-7.08 (m, 4H, H_{Arom}, H_{Imi}), 4.71 (4H, CH₂), 3.88 (bs, 4H, CH₂N), 2.33-2.22 (m, 4H, C═CH₂), 2.01-1.83 (m, 8H, C═CH₂, SiCH), 1.65 (bs, 4H, CH₂), 1.22 (bs, 4H, CH₂), 0.32 (s, 12H, SiCH₃), −0.31 (s, 12H, SiCH₃).

¹³C NMR (CDCl₃, 75.5 MHz) δ 184.9 (Pt-C_{car}), 169.8 (CO), 140.9 (C), 129.2 (CH), 125.1 (CH), 122.0 (C_{Imi}), 121.6 (CH), 120.0 (C_{Imi}), 54.7 (CH₂), 50.1 (CH₂), 41.5 (J ¹³C-¹⁹⁵Pt=158 Hz, SiCH═CH₂), 35.9 (J ¹³C-¹⁹⁵Pt=121 Hz, SiCH═CH₂), 30.6 (CH₂), 26.6 (CH₂), 1.5 (SiCH₃), −1.5 (SiCH₃).

### Example 1: Preparation of [1,1′-(butane-1,4-diyl)bis(3-methylimidazol-2-ylidene)]bis[trans-diiodo(ammonia)platinum] (C1 According to the Invention)

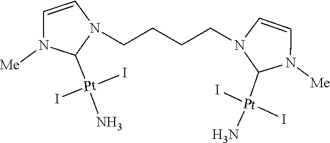

To a solution of [1,1′-(butane-1,4-diyl)bis(3-methylimidazol-2-ylidene)]bis[(1,3-divinyl-1,1,3,3-tetramethyldisiloxane) platinum] (360 mg, 0.37 mmol, 1 equiv.) in toluene (40 mL) at 0° C. under argon was added a solution of iodine (205 mg, 0.81 mmol, 2.2 equiv.) in toluene (15 mL). Then, an aqueous solution of concentrated ammonia (28% NH₃ in H₂O, 255 μL, 1.83 mmol, 5 equiv.) was added at 0° C. and the mixture was slowly allowed to warm to r.t. and stirred for 24 hours. The solvents were removed under reduced pressure and the crude product was purified by recrystallization from dichloromethane/n-heptane to afford the title compound as a pale yellow solid (96 mg, 23% yield).

¹H NMR (CDCl₃, 500.2 MHz) δ 6.86 (s, 2H), 6.83 (s, 2H), 4.73 (bs, 4H), 3.87 (s, 6H), 2.84 (bs, 6H), 2.09 (bs, 4H).

¹³C NMR (CDCl₃, 125.8 MHz) δ 138.9 (C), 122.3 (CH), 120.4 (CH), 49.4 (CH₂), 38.4 (CH₃), 25.8 (CH₂).

Anal. calcd. for C₁₂H₂₄I₄N₆Pt₂, C: 12.53, H: 2.10, N: 7.31; Found, C: 12.67, H: 2.11, N: 7.11.

### Example 2: Preparation of [1,1′-(hexane-1,6-diyl)bis(3-methylimidazol-2-ylidene)]bis[trans-diiodo(ammonia)platinum] (C2 According to the Invention)

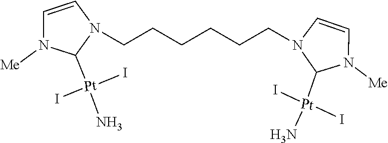

To a solution of [1,1′-(hexane-1,6-diyl)bis(3-methylimidazol-2-ylidene)]bis[(1,3-divinyl-1,1,3,3-tetramethyldisiloxane) platinum] (112 mg, 0.11 mmol, 1 equiv.) in toluene (15 mL) at 0° C. under argon was added a solution of iodine (62.0 mg, 0.24 mmol, 2.2 equiv.) in toluene (5 mL). Then, an aqueous solution of concentrated ammonia (28% NH₃ in H₂O, 75 μL, 0.55 mmol, 5 equiv.) was added at 0° C. and the mixture was slowly allowed to warm to r.t. and stirred for 48 hours. Then silica gel was added and the solvents were removed under reduced pressure to provide a solid sample loading for column chromatography. The crude product was purified over silica gel (eluent: dichloromethane/acetone: 100:0 to 98:2). The pasty solid was taken with diethyl oxide and triturated to provide fine solid in suspension. After decantation, the supernatant liquid was carefully removed and the residue was dried under vacuum to afford the title compound as a pale yellow solid (115 mg, 88% yield).

¹H NMR (Acetone-d₆, 300.2 MHz) δ 7.18 (d, J=2.1 Hz, 2H), 7.13 (d, J=2.1 Hz, 2H), 4.37 (t, J=7.6 Hz, 4H), 3.84 (s, 6H), 3.14 (br, 6H), 2.07-2.04 (m, 4H), 1.51-1.46 (m, 4H).

¹³C NMR (Acetone-d₆, 75.5 MHz) δ 140.5 (C), 122.7 (CHᵢ), 121.3 (CH), 50.7 (CH₂), 38.0 (CH₃), 29.9 (CH₂), 26.6 (CH₂).

IR (v/cm⁻¹) 3309, 3239, 3165, 2925, 2899, 1698, 1606, 1467, 1420, 1253, 1083, 728, 690.

HRMS (ESI+) calcd. for C₁₄H₂₈I₃N₆Pt₂ [M−I]⁺: 1050.8805, found: 1050.8848.

### Example 3: Preparation of [1,1′-(hexane-1-1,6-diyl)bis(3-methylimidazol-2-ylidene)]bis[trans-diiodo(N-cyclohexylamine)platinum] (C3 According to the Invention)

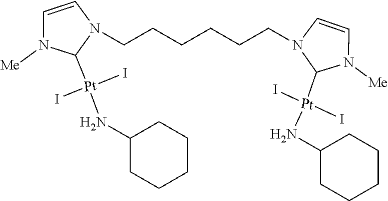

To a solution of [1,1′-(hexane-1,6-diyl)bis(3-methylimidazol-2-ylidene)]bis[(1,3-divinyl-1,1,3,3-tetramethyldisiloxane) platinum] (126 mg, 0.12 mmol, 1 equiv.) in toluene (20 mL) at 0° C. under argon was added a solution of iodine (69.7 mg, 0.26 mmol, 2.2 equiv.) in toluene (5 mL). Then, cyclohexylamine (30 μL, 0.25 mmol, 2 equiv.) was added at 0° C. and the mixture was slowly allowed to warm to r.t. and stirred for 48 hours. Then silica gel was added and the solvents were removed under reduced pressure to provide a solid sample loading for column chromatography. The crude product was purified over silica gel (eluent: dichloromethane/toluene: 80:20). The pasty solid was taken with diethyl oxide and triturated to provide fine solid in suspension. After decantation, the supernatant liquid was carefully removed and the residue was dried under vacuum to afford the title compound as a pale orange solid (96 mg, 57% yield).

¹H NMR (CDCl₃, 500.2 MHz) δ 6.84 (s, 2H), 6.80 (s, 2H), 4.33 (t, J=7.6 Hz, 4H), 3.85 (s, 6H), 3.27-3.22 (m, 2H), 2.95-2.89 (m, 4H), 2.30-2.28 (m, 4H), 2.03-2.00 (m, 4H), 1.79-1.77 (m, 4H), 1.65-1.63 (m, 4H), 1.49-1.47 (m, 4H), 1.35-1.28 (m, 4H), 1.24-1.13 (m, 4H).

¹³C NMR (CDCl₃, 75.5 MHz) δ 138.6 (C), 122.0 (CH), 120.6 (CH), 55.0 (CH), 50.7 (CH₂), 38.2 (CH₃), 36.0 (CH₂), 29.5 (CH₂), 26.2 (CH₂), 25.4 (CH₂), 25.0 (CH₂).

IR (v/cm⁻¹) 3218, 3125, 2925, 2853, 1713, 1571, 1466, 1447, 1376, 1224, 1139, 911, 730, 700.

HRMS (ESI+) calcd. for C₂₆H₄₈I₃N₆Pt₂ [M−I]⁺: 1215.0370, found: 1215.0388.

### Example 4: Preparation of [1,1′-(octane-1,8-diyl)bis(3-methylimidazol-2-ylidene)]bis[trans-diiodo(ammonia)platinum] (C4 According to the Invention)

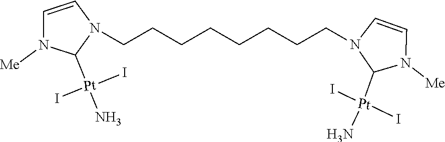

To a solution of [1,1′-(octane-1,8-diyl)bis[(3-methylimidazol-2-ylidene)]bis[(1,3-divinyl-1,1,3,3-tetramethyldisiloxane)platinum] (144 mg, 0.14 mmol, 1 equiv.) in toluene (25 mL) at 0° C. under argon was added a solution of iodine (77 mg, 0.31 mmol, 2.2 equiv.) in toluene (5 mL). Then, an aqueous solution of concentrated ammonia (28% NH₃ in H₂O, 100 μL, 0.70 mmol, 5 equiv.) was added at 0° C. and the mixture was slowly allowed to warm to r.t. and stirred for 48 hours. Then silica gel was added and the solvents were removed under reduced pressure to provide a solid sample loading for column chromatography. The crude product was purified over silica gel (eluent: dichloromethane/acetone: 100:0 to 99:1). The pasty solid was taken with diethyl oxide and triturated to provide fine solid in suspension. After decantation, the supernatant liquid was carefully removed and the residue was dried under vacuum to afford the title compound as a pale yellow solid (123 mg, 73% yield).

¹H NMR (Acetone-d₆, 300.2 MHz) δ 7.16 (d, J=2.1 Hz, 2H), 7.13 (d, J=2.1 Hz, 2H), 4.34 (t, J=7.6 Hz, 4H), 3.83 (s, 6H), 3.13 (br, 6H), 2.07-2.00 (m, 4H), 1.42 (bs, 8H).

¹³C NMR (Acetone-d₆, 75.5 MHz) δ 140.4 (C), 122.5 (CH), 121.2 (C), 50.8 (CH₂), 37.9 (CH₃), 30.0 (CH₂), 29.4 (CH₂), 26.9 (CH₂).

IR (v/cm⁻¹) 3306, 3237, 3164, 3125, 2926, 2854, 1696, 1605, 1466, 1420, 1250, 1047, 690.

HRMS (ESI+) calcd. for C₁₆H₃₂I₃N₆Pt₂ [M−I]+: 1078.9118, found: 1078.9139.

### Example 5: Preparation of [1,1′-(3,5-dioxa-octane-1,8-diyl)bis(3-methylimidazol-2-ylidene)]bis[trans-diiodo(ammonia)platinum] (C5 According to the Invention)

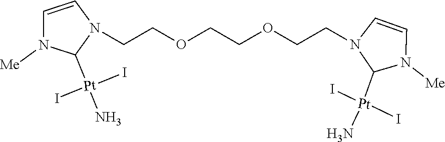

To a solution of 1,1′-(3,5-dioxa-octane-1,8-diyl)bis[(3-methylimidazol-2-ylidene)(1,3-divinyl-1,1,3,3-tetramethyldisiloxane)platinum] (536 mg, 0.51 mmol, 1 equiv.) in toluene (90 mL) at 0° C. under argon was added a solution of iodine (287 mg, 1.13 mmol, 2.2 equiv.) in toluene (15 mL). Then, an aqueous solution of concentrated ammonia (28% NH₃ in H₂O, 150 μL, 1.05 mmol, 2 equiv.) was added at 0° C. and the mixture was slowly allowed to warm to r.t. and stirred for 48 hours. After evaporation of the solvents, the crude product was recrystallized from an ethyl acetate/n-heptane mixture to afford the title compound as a pale yellow solid (504 mg, 81% yield).

¹H NMR (Acetone-d₆, 300.2 MHz) δ 7.17 (d, J=2.0 Hz, 2H), 7.12 (d, J=2.0 Hz, 2H), 4.53 (t, J=5.5 Hz, 4H), 3.99 (t, J=5.5 Hz, 4H), 3.83 (s, 6H), 3.64 (s, 4H), 3.15 (br, 6H).

¹³C NMR (Acetone-d₆, 75.5 MHz) δ 140.6 (C), 123.1 (CH), 122.6 (CH), 71.1 (CH₂), 70.0 (CH₂), 50.9 (CH₂), 38.2 (CH₃).

IR (v/cm⁻¹) 3305, 3240, 3168, 2939, 2868, 1695, 1609, 1463, 1415, 1250, 1111, 1085, 736, 690.

HRMS (ESI+) calcd. for C₁₄H₂₈I₃N₆Pt₂ [M−I]+: 1082.8702, found: 1082.8677.

### Example 6: Preparation of [1,1′-(hexane-1,6-diyl)bis(3-methylimidazol-2-ylidene)]bis[trans-diiodo(N-piperidine)platinum] (C6 According to the Invention)

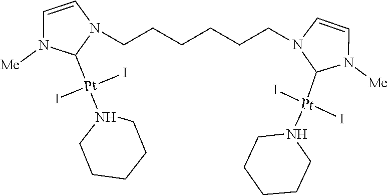

To a solution of 1,1′-(hexane-1,6-diyl)bis[(3-methylimidazol-2-ylidene)(dvtms)]platinum (100 mg, 0.10 mmol, 1 equiv.) in toluene (15 mL) at 0° C. under argon was added a solution of iodine (55 mg, 0.22 mmol, 2.2 equiv.) in toluene (5 mL). Then, piperidine (22 μL, 0.22 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 7:3) to afford the title compound as a pale yellow solid (43 mg, 33% yield).

¹H NMR (CDCl₃, 500.2 MHz) δ 6.82 (d, J=1.8 Hz, 2H), 6.78 (d, J=1.8 Hz, 2Hᵢ), 4.30 (t, J=7.6 Hz, 4H), 3.83 (s, 6H), 3.24-3.16 (m, 4H), 3.11-3.08 (m, 4H), 2.99-2.93 (m, 2H), 2.00 (tt, J=7.6, 7.6 Hz, 4H), 1.76-1.66 (m, 6H), 1.50-1.44 (m, 10H).

¹³C NMR (CDCl₃, 125.8 MHz) δ 138.5 (C), 121.9 (CH), 120.5 (CH), 51.8 (CH₂), 50.7 (CH₂), 38.3 (CH₃), 29.9 (CH₂), 29.4 (CH₂), 26.2 (CH₂), 23.9 (CH₂).

IR (v/cm⁻¹) 3329, 2974, 2928, 2883, 1449, 1417, 1381, 1327, 1275, 1088, 1045, 880.

HRMS (ESI+) calcd. for C₂₄H₄₄I₃N₆Pt₂ [M−I]+: 1187.0057, found: 1187.0024.

### Example 7: Preparation of [1,1′-(hexane-1,6-diyl)bis(3-methylimidazol-2-ylidene)]bis[trans-diiodo(N-morpholine)platinum] (C7 According to the Invention)

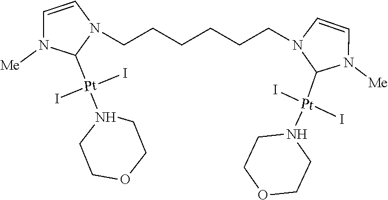

To a solution of [1,1′-(hexane-1,6-diyl)bis(3-methylimidazol-2-ylidene)]bis[(1,3-divinyl-1,1,3,3-tetramethyldisiloxane)platinum] (100 mg, 0.10 mmol, 1 equiv.) in toluene (15 mL) at 0° C. under argon was added a solution of iodine (55 mg, 0.22 mmol, 2.2 equiv.) in toluene (5 mL). Then, morpholine (20 μL, 0.22 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 5:5) to afford the title compound as a pale yellow solid (25 mg, 19% yield).

¹H NMR (CDCl₃, 500.2 MHz) δ 6.83 (d, J=1.9 Hz, 2H), 6.80 (d, J=1.9 Hz, 2H), 4.28 (t, J=7.6 Hz, 4H), 3.83-3.81 (m, 10H), 3.63-3.51 (m, 8H), 3.29-3.25 (m, 2H), 2.91-2.89 (m, 4H), 2.00 (tt, J=7.6, 7.6 Hz, 4H), 1.46 (bs, 4H).

¹³C NMR (CDCl₃, 125.8 MHz) δ 135.1 (C), 122.0 (CH), 120.6 (CH), 68.9 (CH₂), 50.7 (CH₂), 50.6 (CH₂), 38.3 (CH₃), 29.3 (CH₂), 26.2 (CH₂).

IR (v/cm⁻¹) 3327, 2973, 2928, 2883, 1455, 1417, 1380, 1328, 1274, 1088, 1045, 880.

HRMS (ESI+) calcd. for C₂₂H₄₀I₃N₆O₂Pt₂ [M−I]⁺: 1190.9643, found: 1190.9688.

### Example 8: Preparation of {1,1′-(hexane-1,6-diyl)bis[3-(2-hydroxethyl)pimidazol-2-ylidene]}bis[trans-diiodo(ammonia)platinum] (C8 According to the Invention)

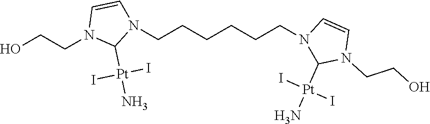

To a solution of {1,1′-(Hexane-1,6-diyl)bis[3-(2-hydroxethyl)imidazol-2-ylidene]}bis[(1,3-divinyl-1,1,3,3-tetramethyldisiloxane)platinum] (80 mg, 0.07 mmol, 1 equiv.) in toluene (10 mL) at 0° C. under argon was added a solution of iodine (41 mg, 0.16 mmol, 2.2 equiv.) in toluene (5 mL). Then, an aqueous solution of concentrated ammonia (28% NH₃ in H₂O, 55 μL, 0.38 mmol, 5 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 3:7) to afford the title compound as a colorless oil (35 mg, 38% yield).

¹H NMR (CD₃OD, 500.2 MHz) δ 7.12 (bs, 2H), 7.08 (bs, 2H), 4.49 (t, J=5.6 Hz, 4H), 4.36 (t, J=7.3 Hz, 4H), 4.05 (t, J=5.6 Hz, 4H), 3.11 (bs, 6H), 2.05 (bs, 4H), 1.48 (bs, 4H).

¹³C NMR (CD₃OD, 125.8 MHz) δ 141.0 (C), 123.1 (CH), 121.7 (CH), 61.7 (CH₂), 53.7 (CH₂), 51.6 (CH₂), 30.4 (CH₂), 27.1 (CH₂).

IR (v/cm⁻¹) 3321, 2944, 2832, 1449, 1416, 1114, 1019.

HRMS (ESI+) calcd. for C₁₆H₃₄I₃N₆O₂Pt₂ [M−I]⁺: 1110.9017, found: 1110.9032.

### Example 9: Preparation of {1,1′-(hexane-1,6-diyl)bis[3-(2-hydroxethyl)imidazol-2-ylidene]}bis{[trans-diiodo(N-cyclohexylamine) platinum]} (C9 According to the Invention)

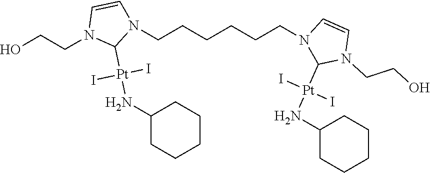

To a solution of {1,1′-(hexane-1,6-diyl)bis[3-(2-hydroxethyl)imidazol-2-ylidene]}bis[(1,3-divinyl-1,1,3,3-tetramethyldisiloxane)platinum] (100 mg, 0.09 mmol, 1 equiv.) in toluene (10 mL) at 0° C. under argon was added a solution of iodine (52 mg, 0.20 mmol, 2.2 equiv.) in toluene (5 mL). Then, cyclohexylamine (25 μL, 0.21 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 4:6) to afford the title compound as a colorless oil (25 mg, 19% yield).

¹H NMR (CDCl₃, 500.2 MHz) δ 6.94 (d, J=1.6 Hz, 2H), 6.87 (d, J=1.6 Hz, 2H), 4.47 (t, J=5.1 Hz, 4H), 4.36 (t, J=7.6 Hz, 4H), 4.26 (td, J=5.6, 5.6 Hz, 4H), 3.26-3.22 (m, 2H), 2.96-2.89 (m, 4H), 2.27-2.25 (m, 4H), 2.01 (bs, 4H), 1.95-1.93 (d, J=5.6 Hz, 2H), 1.79-1.77 (m, 4H), 1.65-1.63 (m, 2H), 1.48 (bs, 4H), 1.35-1.30 (m, 6H), 1.23-1.13 (m, 4H).

¹³C NMR (CDCl₃, 125.8 MHz) δ 138.2 (C), 122.4 (C), 120.4 (C), 60.8 (CH₂), 54.9 (CH), 53.0 (CH₂), 50.9 (CH₂), 35.9 (CH₂), 29.2 (CH₂), 26.1 (CH₂), 25.3 (CH₂), 24.8 (CH₂).

IR (v/cm⁻¹) 3454, 3280, 2924, 2854, 1572, 1461, 1447, 1423, 1358, 1256, 1225, 1141, 1052.

HRMS (ESI+) calcd. for C₃₀H₅₅I₃N₇O₂Pt₂ [M−I+CH₃CN]⁺: 1316.0847, found: 1316.0856.

### Example 10: Preparation of [1,1′-(benzene-1,4-bis-methylenediyl)bis(3-methylimidazol-2-ylidene)]bis[trans-diiodo(N-cyclohexylamine)platinum] (C10 According to the Invention)

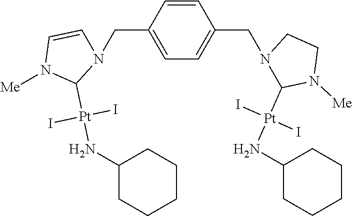

To a solution of 1,1′-(benzene-1,4-bis-methylenediyl)bis[(3-methylimidazol-2-ylidene)(1,3-divinyl-1,1,3,3-tetramethyldisiloxane)platinum] (175 mg, 0.17 mmol, 1 equiv.) in toluene (30 mL) at 0° C. under argon was added a solution of iodine (95 mg, 0.37 mmol, 2.2 equiv.) in toluene (5 mL). Then, cyclohexylamine (40 μL, 0.35 mmol, 2.0 equiv.) was added at 0° C. and the mixture was slowly allowed to warm to r.t. and stirred for 48 hours. . After evaporation of the solvents, the crude product was recrystallized from an ethyl acetate/n-heptane mixture to afford the title compound as a pale orange solid (73 mg, 32% yield).

¹H NMR (Acetone-d₆, 300.2 MHz) δ 7.57 (s, 4H), 7.16 (d, J=2.1 Hz, 2H), 6.94 (d, J=2.1 Hz, 2H), 5.62 (s, 4H), 3.86 (s, 6H), 3.50-3.44 (m, 4H), 3.28-3.22 (m, 2H), 2.36-2.30 (m, 4H), 1.88-1.59 (m, 8H), 1.36-1.22 (m, 8H).

¹³C NMR (Acetone-d₆, 75.5 MHz) δ 141.0 (C), 130.1 (CH), 123.6 (CH), 121.1 (CH), 55.0 (CH), 54.2 (CH₂), 38.2 (CH₃), 36.1 (CH₂), 26.2 (CH₂), 25.6 (CH₂).

IR (v/cm⁻¹) 2931, 2854, 1570, 1464, 1447, 1414, 1229, 1051, 911, 694.

HRMS (ESI+) calcd. for C₃₀H₄₇I₃N₇Pt₂ [M−I+CH₃CN]⁺: 1276.0323, found: 1276.0337.

### Example 11: Preparation of [1,1′-(hexane-1,6-diyl)bis(3-methylbenzo-imidazol-2-ylidene)]bis[trans-diiodo(N-cyclohexylamine)platinum] (C11 According to the Invention)

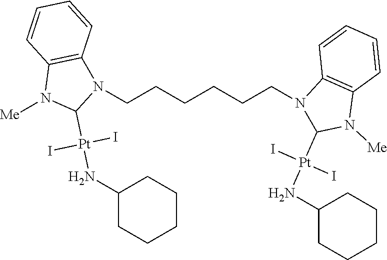

To a solution of [1,1′-(hexane-1,6-diyl)bis(3-methylbenzo-imidazol-2-ylidene)]bis[(1,3-divinyl-1,1,3,3-tetramethyldisiloxane) platinum] (460 mg, 0.41 mmol, 1 equiv.) in a mixture of toluene/dichloromethane 1:1 (60 mL) at 0° C. under argon was added a solution of iodine (210 mg, 0.83 mmol, 2 equiv.) in toluene (10 mL). Then, cyclohexylamine (960 μL, 0.83 mmol, 2 equiv.) was added at 0° C. and the mixture was slowly allowed to warm to r.t. and stirred for 24 hours. Then silica gel was added and the solvents were removed under reduced pressure to provide a solid sample loading for column chromatography. The crude product was purified over silica gel (eluent: dichloromethane/n-heptane: 70:30). The pasty solid was taken with hot ethanol, triturated and filtered to afford the title compound (130 mg, 22% yield).

¹H NMR (CDCl₃, 300.2 MHz) δ 7.37-7.31 (m, 4H), 7.25-7.19 (m, 4H), 4.66 (t, J=7.7 Hz, 4H), 4.07 (s, 6H), 3.36-3.25 (m, 2H), 3.10-2.94 (m, 4H), 2.35-2.94 (m, 4H), 2.22-2.12 (m, 4H), 1.84-1.78 (m, 4H), 1.68-1.61 (m, 4H), 1.41-1.13 (m, 12H).

¹³C NMR (CDCl₃, 75.5 MHz) δ 152.8 (C), 134.3 (C), 122.9 (J ¹³C-¹⁹⁵Pt=7 Hz, CH), 110.3 (J ¹³C-¹⁹⁵Pt=27 Hz, CH), 55.1 (CH), 48.3 (CH₂), 36.1 (CH₃), 34.8 (CH₂), 28.6 (CH₂), 26.9 (CH₂), 25.5 (CH₂), 25.0 (CH₂).

Anal. calcd. for C₃₄H₅₂I₄N₆Pt₂, C: 28.31, H: 3.63, N: 5.83; Found, C: 28.17, H: 3.65, N: 5.45.

### Example 12: Preparation of [1,1′-(hexane-1,6-diyl)bis(3-methylbenzo-imidazol-2-ylidene)]bis[trans-diiodo(N-cyclohexylamine)platinum] (C12 According to the Invention)

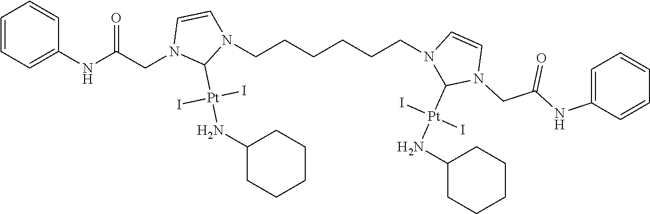

To a solution of {1,1′-(hexane-1,6-diyl)bis[3-(2-oxo-2-(phenylamino)ethypimidazol-2-ylidene]}bis[(1,3-divinyl-1,1,3,3-tetramethyldisiloxane)platinum] (110 mg, 0.09 mmol, 1 equiv.) in toluene (10 mL) at 0° C. under argon was added a solution of iodine (50 mg, 0.19 mmol, 2.2 equiv.) in toluene (5 mL). Then, cyclohexylamine (22 μL, 0.19 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 5:5) to afford the title compound as a colorless oil (96 mg, 69% yield).

¹H NMR (CDCl₃, 500.2 MHz) δ 8.14 (bs, 2H), 7.64 (d, J=8.2 Hz, 4H), 7.30 (t, J=8.2 Hz, 4H), 7.11 (d, J=8.2 Hz, 2H), 7.03 (d, J=1.8 Hz, 2H), 6.96 (d, J=1.8 Hz, 2H), 5.30 (s, 4H), 4.40 (t, J=7.6 Hz, 4H), 3.29-3.22 (m, 2H), 3.03-2.97 (m, 4H), 2.27-2.25 (m, 4H), 2.06 (bs, 4H), 1.79-1.77 (m, 4H), 1.67-1.62 (m, 2H), 1.51 (bs, 4H), 1.37-1.29 (m, 6H), 1.22-1.12 (m, 4H).

¹³C NMR (CDCl₃, 125.8 MHz) δ 165.0 (CO), 141.4 (C), 137.5 (C), 129.0 (CH), 124.9 (CH), 122.3 (CH), 120.9 (CH), 120.5 (CH), 55.5 (CH₂), 55.2 (CH₂), 51.0 (CH₂), 36.0 (CH₂), 29.3 (CH₂), 26.2 (CH₂), 25.4 (CH₂), 25.0 (CH₂).

IR (v/cm⁻¹) 3304, 3053, 2895, 2938, 2860, 1691, 1600, 1535, 1498, 1444, 1421, 1264, 1051, 908, 895.

HRMS (ESI+) calcd. for C₄₂H₆₁I₃N₉O₂Pt₂ [M−I+CH₃CN]⁺: 1494.1378, found: 1494.1389.

### Example 13: {1,1-(Hexane-1,6-diyl)bis[3-(2-hydroxethyl)imidazol-2-ylidene]}bis[trans-diiodo(N-piperidine)platinum] (C13 According to the Invention)

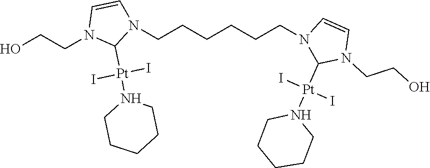

To a solution of {1,1′-(hexane-1,6-diyl)bis[3-(2-hydroxethyl)imidazol-2-ylidene]}bis[(1,3-divinyl-1,1,3,3-tetramethyldisiloxane)platinum] (100 mg, 0.09 mmol, 1 equiv.) in toluene (10 mL) at 0° C. under argon was added a solution of iodine (52 mg, 0.20 mmol, 2.2 equiv.) in toluene (5 mL). Then, piperidine (20 μL, 0.21 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 4:6) to afford the title compound as a colorless oil (21 mg, 16% yield).

¹H NMR (CDCl₃, 500.2 MHz) δ 6.92 (d, J=1.9 Hz, 2H), 6.85 (d, J=1.9 Hz, 2H), 4.43 (t, J=4.9 Hz, 4H), 4.32 (t, J=7.5 Hz, 4H), 4.26 (t, J=4.9 Hz, 4H), 3.26-3.13 (m, 4H), 3.09-3.06 (m, 4H), 2.99-2.93 (m, 2H), 2.01 (bs, 4H), 1.76-1.66 (m, 6H), 1.52-1.43 (m, 10H).

¹³C NMR (CDCl₃, 125.8 MHz) δ 135.7 (C), 122.4 (CH), 120.3 (CH), 60.7 (CH₂), 53.1 (CH₂), 51.8 (CH₂), 50.9 (CH₂), 29.2 (CH₂), 28.3 (CH₂), 26.1 (CH₂), 23.7 (CH₂).

HRMS (ESI+) calcd. for C₂₆H₄₉I₄N₆O₂Pt₂ [M+H]⁺: 1374.9391, found: 1374.9371.

### Example 14: {1,1-(Hexane-1,6-diyl)bis[3-(2-hydroxethyl)imidazol-2-ylidene]}bis[trans-diiodo(N-morpholine)platinum] (C14 According to the Invention)

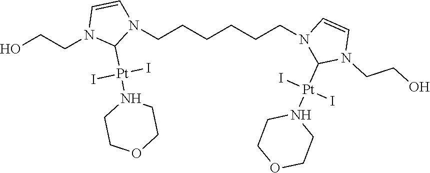

To a solution of {1,1′-(hexane-1,6-diyl)bis[3-(2-hydroxethyl)imidazol-2-ylidene]}bis[(1,3-divinyl-1,1,3,3-tetramethyldisiloxane)platinum] (100 mg, 0.09 mmol, 1 equiv.) in toluene (10 mL) at 0° C. under argon was added a solution of iodine (52 mg, 0.20 mmol, 2.2 equiv.) in toluene (5 mL). Then, morpholine (18 μL, 0.21 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 4:6) to afford the title compound as a colorless oil (36 mg, 28% yield).

¹H NMR (CDCl₃, 500.2 MHz) δ 6.96 (d, J=1.9 Hz, 2H), 6.87 (d, J=1.9 Hz, 2H), 4.44 (t, J=5.2 Hz, 4H), 4.31 (t, J=7.2 Hz, 4H), 4.24 (bs, 4H), 3.84-3.82 (m, 4H), 3.62-3.51 (m, 8H), 3.30-3.26 (m, 2H), 2.90-2.88 (m, 4H), 2.02 (bs, 4H), 1.47 (bs, 4H).

¹³C NMR (CDCl₃, 125.8 MHz) δ 134.5 (C), 122.7 (CH), 120.7 (CH), 68.9 (CH₂), 60.9 (CH₂), 53.3 (CH₂), 51.2 (CH₂), 50.9 (CH₂), 29.3 (CH₂), 26.3 (CH₂).

IR (v/cm⁻¹) 3452, 3203, 3130, 2926, 2853, 1462, 1447, 1421, 1251, 1226, 1191, 1117, 1089, 1063, 1031, 881.

HRMS (ESI+) calcd. for C₂₄H₄₅I₄N₆O₂Pt₂ [M+H]⁺: 1378.8876, found: 1378.8839.

### Example 15: [1,1′-(Hexane-1,6-diyl)bis(3-methylimidazol-2-ylidene)]bis[trans-diiodo(N-4-trifluoromethylpyridine)platinum] (C15 According to the Invention)

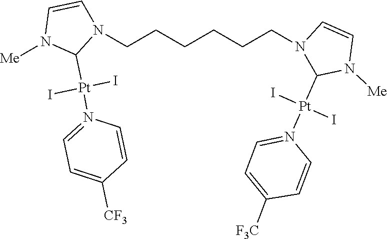

To a solution of [1,1′-(hexane-1,6-diyl)bis(3-methylimidazol-2-ylidene)]bis[(1,3-divinyl-1,1,3,3-tetramethyldisiloxane)platinum] (100 mg, 0.10 mmol, 1 equiv.) in toluene (15 mL) at 0° C. under argon was added a solution of iodine (55 mg, 0.22 mmol, 2.2 equiv.) in toluene (5 mL). Then, 4-trifluoromethylpyridine (25 μL, 0.22 mmol, 2.2 equiv.) was added at 0° C. and the mixture was slowly allowed to warm to r.t. and stirred for 18 hours. Then silica gel was added and the solvents were removed under reduced pressure to provide a solid sample loading for column chromatography. The crude product was purified over silica gel (eluent: n-heptane/EtOAc 6:4) to afford the title compound as a pale yellow solid (72 mg, 51% yield).

¹H NMR (CDCl₃, 500.2 MHz) δ 9.31 (d, J=5.8 Hz, 4H), 7.55 (d, J=5.8 Hz, 4H), 6.89 (d, J=1.8 Hz, 2H), 6.83 (d, J=1.8 Hz, 2H), 4.44 (t, J=7.6 Hz, 4H), 3.95 (s, 6H), 2.13 (tt, J=7.6, 7.6 Hz, 4H), 1.58 (bs, 4H).

¹³C NMR (Acetone-d₆, 175.8 MHz) δ 156.0 (CH), 139.3 (J ¹³C-¹⁹F=35 Hz, C), 134.5 (C), 123.3 (CH), 123.4 (J ¹³C-¹⁹F=273 Hz, CF₃), 122.0 (CH), 121.9 (CH), 51.2 (CH₂), 38.4 (CH₃), 30.0 (CH₂), 26.7 (CH₂).

¹⁹F NMR (CDCl₃, 282.4 MHz) δ -65.2 (CF₃).

IR (v/cm⁻¹) 3104, 2926, 2855, 1469, 1419, 1323, 1226, 1179, 1143, 1101, 1060, 836.

HRMS (ESI+) calcd. for C₂₆H₃₀F₆I₃N₆Pt₂ [M−I]⁺: 1310.8866, found: 1310.8923.

### Example 16: Preparation of trans-Diiodo(ammonia)(1,3-dimethylimidazol-2-ylidene)platinum (MS113)

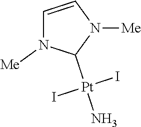

To a solution of [(1,3-dimethylimidazol-2-ylidene)(dvmts)]platinum (Berthon-Gelloz, G.; Buisine, O.; Brière, J.-F.; Michaud, G.; Stérin, S.; Mignani, G.; Tinant, B.; Declercq, J.-P.; Chapon, D.; Markó, I. E. J. Organomet. Chem. 2005, 690, 6156-6168) (52 mg, 0.11 mmol, 1 equiv.) in toluene (15 mL) at 0° C. under argon was added a solution of iodine (30.4 mg, 0.12 mmol, 1.1 equiv.) in toluene (5 mL). Then, an aqueous solution of concentrated ammonia (28% NH₃ in H₂O, 35 μL, 0.26 mmol, 2.4 equiv.) was added at 0° C. and the mixture was slowly allowed to warm to r.t. and stirred for 24 hours. Then silica gel was added and the solvents were removed under reduced pressure to provide a solid sample loading for column chromatography. The crude product was purified over silica gel (eluent: n-heptane/EtOAc 7:3) to afford the title compound as a pale yellow solid (44 mg, 72% yield).

¹H NMR (CDCl₃, 300.2 MHz) δ 6.79 (s, 2H, H_{Imi}), 3.87 (s, 6H, NCH₃), 2.60 (bs, 3H, NH₃).

¹³C NMR (CDCl₃, 75.5 MHZ) δ 137.6 (Pt-C_{car}), 121.9 (C_{Imi}), 38.2 (NCH₃).

Anal. calcd. for C₅H₁₁I₂N₃Pt, C: 10.68, H: 1.97, N: 7.48; Found, C: 11.27, H: 1.75, N: 7.09.

### Example 17: Preparation of trans-Diiodo(N-cyclohexylamine)(1,3-dimethylimidazol-2-ylidene)platinum (MS140)

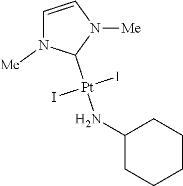

To a solution of [(1,3-dimethylimidazol-2-ylidene)(dvmts)]platinum (Berthon-Gelloz, G.; Buisine, O.; Brière, J.-F.; Michaud, G.; Stérin, S.; Mignani, G.; Tinant, B.; Declercq, J.-P.; Chapon, D.; Markó, I. E. J. Organomet. Chem. 2005, 690, 6156-6168) (310 mg, 0.65 mmol, 1 equiv.) in toluene (40 mL) at 0° C. under argon was added a solution of iodine (181 mg, 0.71 mmol, 1.1 equiv.) in toluene (15 mL). Then, cyclohexylamine (75 μL, 0.65 mmol, 1 equiv.) was added at 0° C. and the mixture was slowly allowed to warm to r.t. and stirred for 24 hours. Then silica gel was added and the solvents were removed under reduced pressure to provide a solid sample loading for column chromatography. The crude product was purified over silica gel (eluent: n-heptane/EtOAc 7:3) to afford the title compound as a pale yellow solid (270 mg, 65% yield).

¹H NMR (CDCl₃, 300.2 MHz) δ 6.79 (s, 2H, H_{Imi}), 3.85 (s, 6H, NCH₃), 3.30-3.20 (m, 1H, NCH), 2.98-2.82 (m, 2H, NH₂Cy), 2.31-2.27 (m, 2H, Cy), 1.80-1.61 (m, 4H, Cy), 1.40-1.11 (m, 4H, Cy).

¹³C NMR (CDCl₃, 75.5 MHZ) δ 139.6 (Pt-C_{car}), 121.6 (C_{Imi}), 55.0 (NCH), 38.1 (NCH₃), 36.1 (Cy), 25.5 (Cy), 25.0 (Cy).

Anal. calcd. for C₁₁H₂₂I₂N₃Pt, C: 20.51, H: 3.29, N: 6.52; Found, C: 20.79, H: 3.21, N: 6.27.

### Biological Results

### Material and Methods

Cell culture. The A2780 ovarian carcinoma cell lines were purchased from ECACC (Salisbury, UK) and were grown in complete RPMI medium supplemented with 10% fetal calf serum, in the presence of penicillin, streptomycin. The resistance of A2780/DDP cells to cisplatin was maintained by monthly treatment with 1 μM cisplatin for 4 days. H1299 Non Small Cell Lung Carcinoma (ATCC® CRL-5803™) and were grown in RPMI medium supplemented with 10% fetal calf serum, 1% HEPES, 1% Sodium Pyruvate in the presence of penicillin and streptomycin. In addition, four cervical cancerous cell lines, already resistant to some conventional chemotherapy (Gemcitabin, methothrexate, vinorelbin and/or cisplatin) were chosen: IC5 (from Institut Curie), CRL1550 (ATCC® CRL1550™), CC11⁺ (Cellosaurus CVCL_DF82) and CRL7920 (ATCC® CRL7920™). They were grown in complete RPMI medium supplemented with 10% fetal calf serum, in the presence of penicillin, streptomycin.

Cell proliferation evaluation. Cells were treated with various concentrations of NHC-Pt, at 37° C. under humidity and 5% CO₂ conditions for 96 h. Cellular growth was quantified using the Moxi Z Mini automated cell counter (Orflox technology). CellTiter Glo® Luminescent Cell Viability Assay following the kit instructions (Promega) was also used. But since doses that inhibit 50% of cell proliferation (IC₅₀) not always correlated to the number of cells, as previously observed (Uehara, T., Mitsuhashi, A., Tsuruoka, N., and Shozu, M. (2015) Metformin potentiates the anticancer effects of cisplatin under normoxic conditions in vitro, Oncology reports 33, 744-750), cell counting was privileged.

### Radiosensitization Assay

For one day or 10 min pretreatment, cells were seeded at a number allowing 5 doubling populations growth without reaching confluence in the untreated samples. They were then pretreated during the time indicated at the indicated NHC-Pt concentrations, irradiated at 1, 2, 3 and 4 Gy for A2780 cell lines and 2. 4. 6 and 8 Gy for H1299 and allow growing until 6 days. IC5 cells were irradiated at 1, 2, 3, 4 and 5 Gy, CC11⁺ were irradiated at 2, 3, 4, 5 and 6 Gy and allow growing until 7 days, CRL1550 were irradiated at 2, 4, 6 and 8 Gy and allow growing until 5 days. CRL79200 were irradiated at 1, 2, 3 and 4 Gy and allow growing until 15 days. For 4 days pretreatment, cells were seeding in the conditions of cell cytotoxicity determination, trypsinised, seeded at a number allowing 5 doubling population growth without reaching confluence in the untreated samples and treated one day before irradiation. Irradiation is using GSR D1 irradiator (gamma-ray, 662 keV). The cells were left to grow for 6 days to allow at least 5 population doublings. Cell count of each well is taken and a graph is plotted with the percentage survival of each treatment of cells with its control (0 Gy).

The survival plot is made in a KaleidaGraph software where the linear quadratic fit model: S(D)/S(0)=exp(−αD−βD2) is used to make the curve. The D10 value (dose of Gy at which only 10% cells survive) is noted from the curve.

Immunofluorescence Assays. A2780 cells were plated on coverslips in 6-well plates. After 1 day of incubation with the various NHC-Pt at their RS doses, cells were irradiated at 2 Gys, incubated the time indicated post-irradiation, washed with phosphate-buffered saline (PBS), then fixed 10 minutes in 4% formaldehyde. After a wash with PBS, cells were permeabilised 2 min using 0.5% Triton X-100 and is washed with PBS. The cells were incubated in blocking buffer (5% bovine serum albumin in PBS) for 30 min before being incubated for 1 h with the primary mouse monoclonal antibody against g-H2AX (milipore) or 53BP1. After three washes with PBS, the cells were incubated for an additional 1 h with the Alexa Fluor 488-conjugated secondary antibody (Alexa Fluor 488 goat anti-mouse IgG; Life Technologies). Nuclei were labeled using DAPI and the coverslides were mounted with Vectashield™. Acquisitions were performed on a3D-dev or 3D-SIM in the microscopy platform from Institut Curie. ImageJ software (NCBI) was used to project the z-stacks and count the number of g-H2AX or 53BP1 spots as well as their intensity and size in nuclei.

### Results and Discussions

### Bimetallic Platinum-NHC Complexes

As mentioned above, the synthesis of the complexes according to the invention involves two main steps: first coordination of an NHC to a Pt⁽⁰⁾(dvtms) derivative (Berthon-Gelloz, G., et al. (2005) Synthetic and structural studies of NHC-Pt(dvtms) complexes and their application as alkene hydrosilylation catalysts (NHC=N-heterocyclic carbene, dvtms=divinyltetramethylsiloxane), Journal of Organometallic Chemistry 690, 6156-6168), and subsequent oxidation of Pt⁽⁰⁾ into Pt^{(II)} by addition of iodine in the presence of the desired amine.

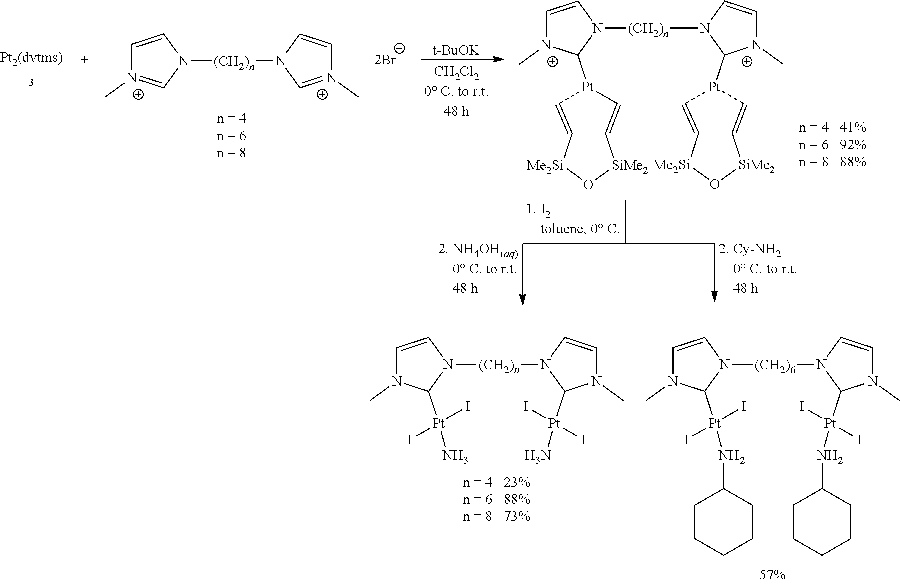

Other platinum complexes used according to the present invention are the monometallic complexes MS113 and MS140 (WO2009/118475) (Examples 16 and 17).

The antiproliferative and radiosensibilizing activities of complexes C1-C5, MS140 and MS113 have been investigated as shown hereafter.

### Antiproliferative Properties of the NHC-Pt

The inventors first established the antiproliferative activities of two mono- and four bi-metallic NHC-Pt complexes on ovarian A2780 and non-small lung carcinoma (NSLC) H1299 cancerous cell lines since both cell lines are presentative of the cancer cell lines treated of first instance with a chemotherapy based on platinum complexes (Muggia, F. M., Garcia Jimenez, M., and Murthy, P. (2019) Platinum compounds: Their continued impact on ovarian cancer treatment, Inorg Chim Acta 496, 119037) and radiotherapy and are widely for cell culture experiments. Moreover the H1299 cell line was shown to be more radioresistant than A2780 cell lines (D10 of 3.3 versus 9.7) affording to test radiosensitizing ability of our complexes in cell lines presenting various radiosensitivity degrees. In addition, A2780cis which is resistant to the anti-tumor drug cisplatin was chosen to detect if the new complexes overcome the resistance to cisplatin. All cell lines have been treated for 96 h with increasing doses of the mono and bi-metallic complexes. In A2780 cell lines, the bi-metallic complexes gave the same results as previously found for other bi-metallic complex from the NHC-Pt series (IC₅₀ around 1-2 μM)(Chtchigrovsky, M., Eloy, L., Jullien, H., Saker, L., Segal-Bendirdjian, E., Poupon, J., Bombard, S., Cresteil, T., Retailleau, P., and Marinetti, A. (2013) Antitumor trans-N-heterocyclic carbene-amine-Pt(II) complexes: synthesis of dinuclear species and exploratory investigations of DNA binding and cytotoxicity mechanisms, J. Med. Chem. 56, 2074-2086) showing no influence of the chain site attachment on antiproliferative properties. Considering the length chain, the C1 seems 3 times more potent (Table 1). For comparison IC₅₀ of cisplatin has been evaluated at 0.3 μM.

Concerning the two mononuclear complexes, they show less efficient cytotoxicity than the previous mononuclear complexes which IC₅₀ were around 0.5 μM, suggesting a marked influence of the side chain (presence of two cyclo-hexyl of one cyclo-hexy+butyl chain) on the anti-proliferative effect. In addition, the bi-metallic complexes exhibit higher cytotoxicity than the mono-metallic complexes, C1 being the more potent one. The same trend has been observed in H1299 cell line (Table 1).

Interestingly, we also performed the proliferation assays following seven days treatment because these are the cell culture conditions used in our irradiation assays and IC₅₀ are similar to the ones found for the four days treatments, indicating that increasing incubation time up to 4 days does not change the proliferation inhibition efficiency of the NHC-Pt complexes. Interestingly, all NHC-Pt complexes overcome the resistance to cisplatin in H1299 since their IC₅₀ values were lower than the ones of cisplatin. It has been shown that whereas C2 is able to counteract the cisplatin resistance in A780cis cell line (IC₅₀ of 2 μM versus 6 μM), it is not the case of MS113.

Concerning the cervical cancerous cell lines, the inventors confirmed that C2 and MS113 are still cytotoxic in the four selected cell lines showing the same trend than in A2780 and H1299 cell lines, that is a higher efficiency for C2 (1.2 to 1.6-fold as compared to MS113). The new complexes (second generation of NHC-Pt complexes from C5 to C9) also display efficient cytotoxic activities among the different cell lines C7≥C9>C2>C6>C5>C8 with IC50 comprised between 0.58 μM (for C7 and C9 in IC5 for example) and 8.5μM for C8 in IC5. It can also be noted that IC5 and CC11+ are more sensitive to the various complexes than CRL1550 and CRL7920. In IC5, C7 and C9 display the same cytotoxicity than cisplatin whereas for the other complexes and in CRL1550 and CC11⁺ cisplatin remains more efficient.

TABLE 1
IC₅₀ (μM) of the Pt-NHC complexes on A2780 and H1299 after
4 days and 7 days treatments, in comparison to cisplatin

| | A2780 | A2780 | H1299 | H1299 |
|---|---|---|---|---|
| NHC-Pt | 4 days | 7 days | 4 days | 7 days |
| MS113 | 4.2 ± 0.2 | 4.5 ± 0.2 | 2.4 ± 0.5 | 5.62 ± 0.5 |
| MS140 | 2.86 ± 1.09 | 2.6 ± 0.8 | 1.85 ± 0.2 | 1.61 ± 0.3 |
| C1 | 0.8 ± 0.2 | 1.5 ± 0.05 | 0.97 ± 0.2 | ND |
| C2 | 1.60 ± 0.15 | 1.6 ± 0.02 | 1.29 ± 0.2 | 1.46 ± 0.3 |
| C3 | 1.77 ± 0.09 | 1.89 ± 0.02 | 1.73 ± 0.2 | ND |
| C4 | 1.68 ± 005 | 1.86 ± 0.8 | 1.08 ± 0..2 | 2.56 ± 0.4 |

TABLE 2
IC₅₀ (μM) of the Pt-NHC complexes on IC5, CC11⁺, CRL1550 and
CRL7920 after 4 days treatments, in comparison to cisplatin

| NHC-Pt | IC5 | CC11⁺ | CRL1550 | CRL7920 |
|---|---|---|---|---|
| MS113 | 4.31 ± 0.3 | 2.64 ± 0.3 | 4.84 ± 0.5 | 5.85 ± 0.5 |
| C2 | 2.78 ± 0.2 | 2.25 ± 0.2 | 2.95 ± 0.2 | 4.12 ± 0.4 |
| C5 | 3.8 ± 0.3 | 3.87 ± 0.3 | 5.6 ± 0.5 | 6.14 |
| C6 | 2.99 ± 0.3 | 4.49 ± 0.5 | 5.04 ± 0.5 | ND |
| C7 | 0.62v ± 0.06 | 0.94 ± 0.09 | ND | ND |
| C8 | 8.49v ± 0.8 | 5.46 ± 0.5 | ND | ND |
| C9 | 0.58 ± 0.06 | ND | ND | ND |
| Cisplatin | 0.49 ± 0.05 | 0.006 | 0.39 ± 0.04 | ND |

### Radiosensitising Properties

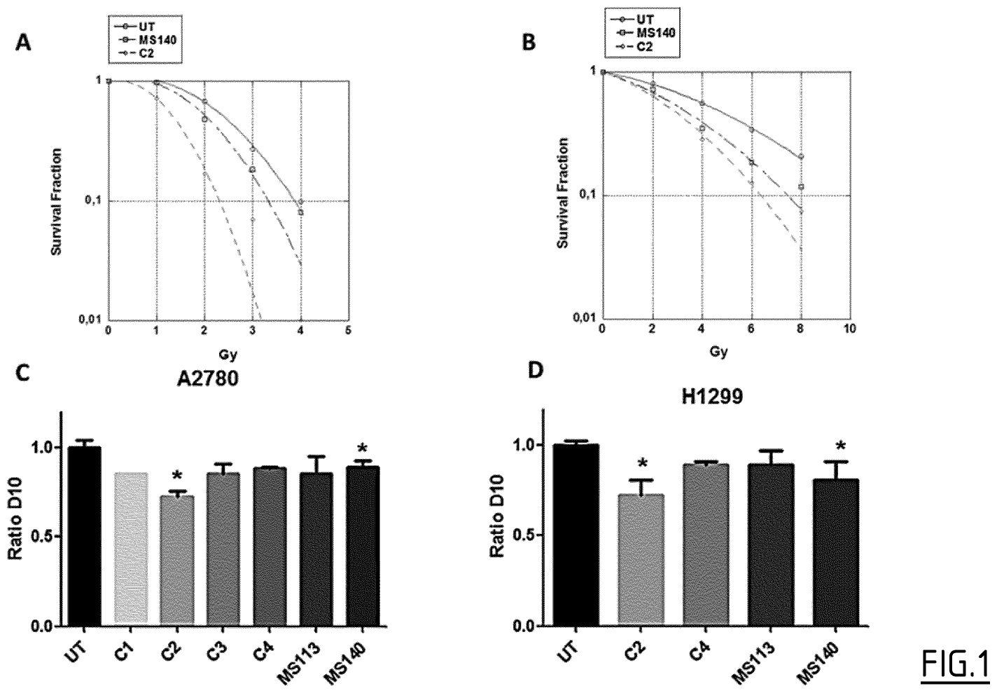

All the complexes were first evaluated for their radiosensitizing properties on A2780 and H1299 cell lines. Cells were treated by increasing irradiation doses in combination with 1 μM complexes, doses that do not induce more than 10% inhibition proliferation in absence of irradiation. This procedure was chosen in order to detect the potential synergistic effect of the complexes only in conditions where their intrinsic cytotoxicity has been minimized. Cells were pretreated one day before irradiation, irradiated at the doses indicated in the experimental part, depending on the sensitivity of the cells to ionizing radiations. The D10 doses were evaluated as the doses allowing 10% survival six days post-irradiation. Of note, the D10 value for A2780 is 3.5±0.2 Gy, while the one of H1299 is 9.7±0.3 indicating that the NSCLC cell line is radioresistant as compared to A2780. The cervical cancerous cell lines display also various radio-sensitivities with D10 value of 2.3 for CRL7920, 4.5 for IC5, 4.6 for CC11+ and 6.9 for CRL1550. The radiosensitizing effect of the complexes was reflected by the drop of the ionizing radiation dose required to induce the 10% cell proliferation as compared to the irradiation dose in the absence of complexes. The D10 values of the irradiation assays performed in the presence of complexes where rationalized to the one performed in the absence of complexes and the D10 ratio are represented in FIGS. 1-3 for the mono- and bi-metallic complexes in both cell lines.

As shown in FIGS. 1A-D, only MS140 and C2 display significant radiosensitizing properties at 1 μM concentration in both cell lines.

Irradiation assays were performed with increasing concentrations of complexes at doses that induce from 10 to 50% cell proliferation inhibition in the assays in absence of irradiation. Interestingly, in these conditions, all complexes induce radiosensitizing effect, but some differences can be noted according to the cell lines and to the complexes.

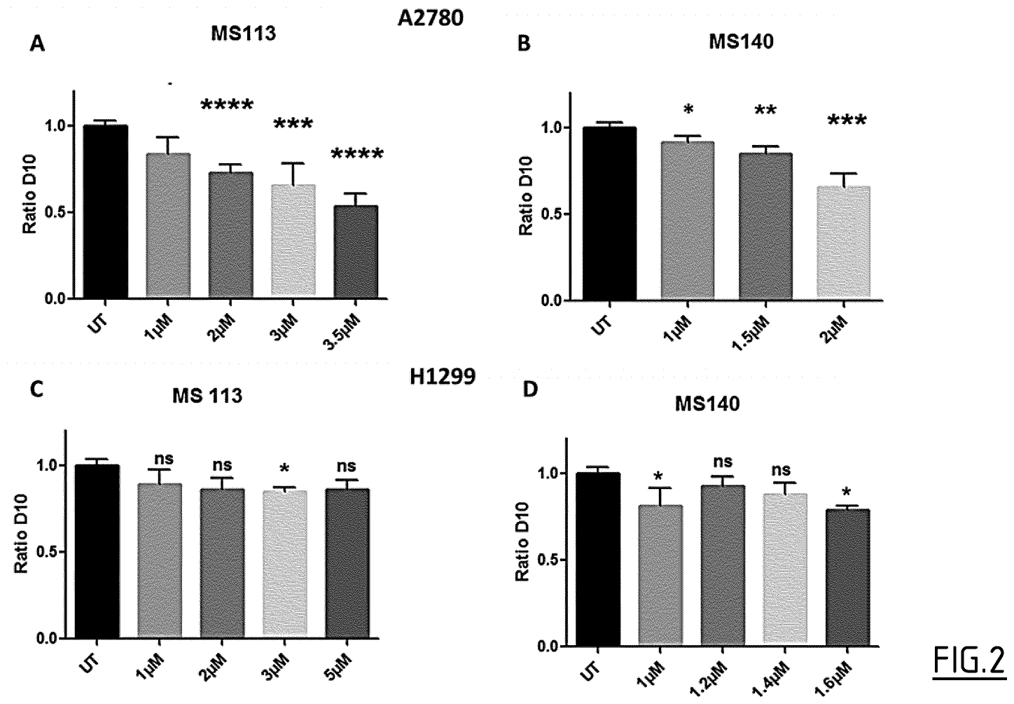

The mono-metallic complexes reduced the D10 more efficiently in the radiosensitive cell line than in the radioresistant cell line (FIG. 2). In A2780, the D10 ratio is reduced until 0.55 and 0.64 for MS140 and MS113, respectively, while in H1299 cell line, the maximum reduction reaches 0.7 and 0.75 for MS140 and MS113, respectively. However, in both cell lines the effective dose is lower for MS140 than MS113 that could reflect their IC₅₀ (Table 1). The decrease of D10 is clearly dose dependent in A2780 whereas it is less pronounced in H1299. MS113 has not been yet evaluated in the four cervical cancerous cell lines.

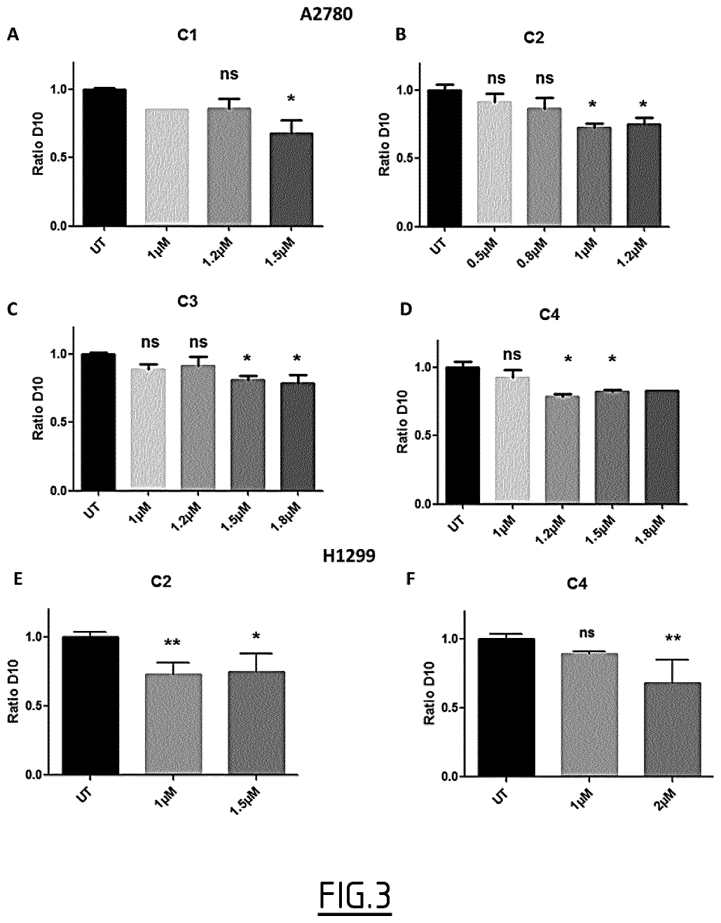

The bimetallic complexes induce also a dose depend reduction of the D10 ratio values until 0.66, 0.72, 0.78 and 0.81 for complex C1, C2, C3 and C4, respectively in A2780 (FIG. 3). In H1299, the two representative complexes C2 and C4 display also a D10 ratio value not exceeding 0.74 for complex C2 and 0.81 for complex C4.

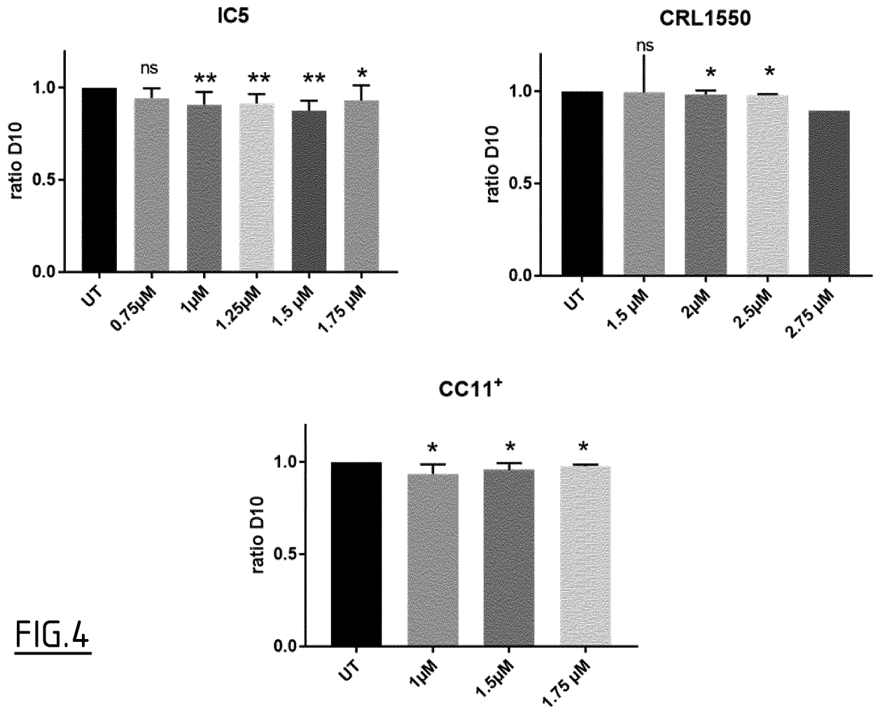

C2 was then evaluated in three the cervical cancerous cell lines, IC5, CC11³⁰ and CRL1550 at doses that induce from 10 to 50% cell proliferation inhibition in the assays in absence of irradiation. This complex still shows RS properties in a dose dependent manner in each cell line (FIG. 4). Of note, the D10 ratio value is reduced until 0.82, 0.89 and 0.92 in IC5, CRL1550 and CC11⁺, respectively. The evaluation of the second generation of complexes is ongoing. Preliminary data indicate a potent RS property for some of them.

### Analysis of the Mechanism of Action of the Radiosensitizing Properties of Metallic Complexes

The treatment conditions were modified in order to appreciate the requirements for the radiosensitzing effect that are the incubation time pre-irradiation that will influence the amount of platinum entering cells and consequently bound to DNA (Chtchigrovsky, M., Eloy, L., Jullien, H., Saker, L., Segal-Bendirdjian, E., Poupon, J., Bombard, S., Cresteil, T., Retailleau, P., and Marinetti, A. (2013) Antitumor trans-N-heterocyclic carbene-amine-Pt(II) complexes: synthesis of dinuclear species and exploratory investigations of DNA binding and cytotoxicity mechanisms, J. Med. Chem. 56, 2074-2086) and the presence of the complexes post-irradiation. In A2780 cell line, the pre-incubation time was increased up to 4 days to optimize the cell uptake of the complexes and their binding to DNA or the complexes were removed post-irradiation. The irradiation assays were performed at the respective concentrations of the complexes inducing radiosensitizing effect determined from FIGS. 2 and 3). For all complexes, the radiosensitizing effect is independent of the pre-incubation time (1 day versus 4 days) and of presence of the complex in the medium post-irradiation suggesting that the amount of platinum complex entering cells and bound to DNA during one day incubation pre-irradiation, is sufficient for this effect.

Since NHC-Pt complexes are known to bind to DNA by coordination (Betzer, J. F., Nuter, F., Chtchigrovsky, M., Hamon, F., Kellermann, G., Ali, S., Calmejane, M. A., Roque, S., Poupon, J., Cresteil, T., Teulade-Fichou, M. P., Marinetti, A., and Bombard, S. (2016) Linking of Antitumor trans NHC-Pt(II) Complexes to G-Quadruplex DNA Ligand for Telomeric Targeting, Bioconjug Chem 27, 1456-1470; Brissy, D., Skander, M., Retailleau, P., and Marinetti, A. (2007) N-Heterocyclic Carbenes in the Synthesis of Axially Chiral Square-Planar Platinum Complexes, Organometallics 26, 5782-5785) and that radiosensitizing effect can be induced by a delay in IR-induced DNA damage (Sears, C. R., Cooney, S. A., Chin-Sinex, H., Mendonca, M. S., and Turchi, J. J. (2016) DNA damage response (DDR) pathway engagement in cisplatin radiosensitization of non-small cell lung cancer, DNA Repair (Amst) 40, 35-46) a kinetic study of the repair of these damages was performed in A2780 cell lines treated by MS113, MS140 or C2 in combination with irradiation.

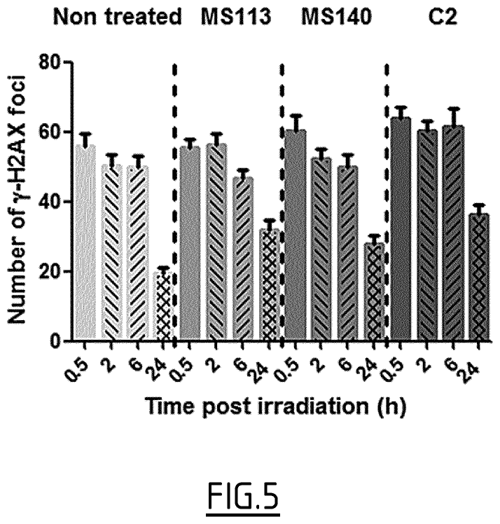

The inventors analyzed, by immunofluorescence, the γ-H2AX and 53BP1 foci, γ-H2AX being a DNA damage sensor and 53BP1 being a DNA repair sensor at different time post-irradiation (0.5-24 h). The results in FIG. 5 clearly show that only complex C2 induced a delay in the DNA damage repair 2 h and 6 h post irradiation.

Thus the inventors have shown that the NHC-Pt complexes according to the invention display high cytotoxicity in three cell lines and they are able to overcome the cisplatin resistance in the NSCLC H1299 cell line (and some in ovarian resistant A2780cis). All complexes show radiosensitizing (RS) properties in both the radiosensitive A2780 and the radioresistant H1299 cancerous cell lines in a concentration dependent manner.

One mono- and one bi-metallic (C2) complexes were revealed to be more potent since they display their RS activity from 1 μM dose.

Of note, the RS properties of the representative complex C2 has been confirmed in three cervical cancerous cell lines.

A set of complexes of second generation (C5-C9 derived from C2) have been synthetized and two of them (C7 and C9) show improved cytotoxic activity as compared to C2 in three cervical cancerous cell lines.

For all complexes, the window of concentration range of the complexes allowing RS without affecting cell proliferation more than 50% in absence of irradiation is very narrow: 1 to 1.8 μM for bi-metallic complexes and MS140 and 2-3.5 μM for MS113. The inventors confirmed the same RS properties of C2 in three cervical cancerous cell lines at concentrations.

In addition to the concentration range requirement for RS, it was shown for all complexes that one day pre-incubation is sufficient and that their presence post-irradiation is not mandatory.

1. A radiosensitizer comprising a mono- or bimetallic (Amine)Platinum(II) N-Heterocyclic Carbene complex having the following formula (I1): 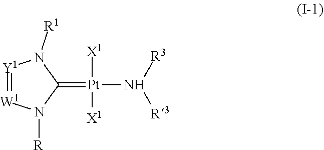
wherein:
R is a group having the below formula (I′): 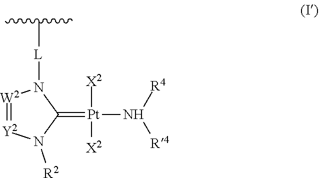
or R is selected from the group consisting of: a C₁-C₆ alkyl group, a C₃-C₆ cycloalkyl, a C₆-C₁₀ aryl, and a (C₆-C₁₀)aryl(C₁-C₆)alkyl group;
L is a linker selected from the group consisting of: a C₁-C₁₂ alkanediyl group, a phenylene-bis(alkanediyl) group, a biphenyldiyl-bis(alkanediyl) group, and an heteroarylidene-bis(alkanediyl) group, said alkanediyl, phenylene and heteroarylidene groups being possibly substituted with one or several substituents such as C₁-C₆ alkyl groups, C₅-C₁₀ aryl groups, heteroaryl, and (hetero)cycloalkyl groups,
wherein said alkanediyl groups may be interrupted with one or several heteroatoms;
X¹ and X², identical or different, are selected from the group consisting of: iodide, bromide, chloride, and nitrato (ONO₂);
Y¹ and Y², identical or different, are either a C—R⁵ group or a N atom, R⁵ being selected from the group consisting of: H, a C₁-C₆ alkyl group, C₃-C₆ cycloalkyl group, and an optionally substituted phenyl group,
W¹ and W², identical or different, are either a C—R⁶ group or a N atom, R⁶ being selected from the group consisting of: H, a C₁-C₆ alkyl group, C₃-C₆ cycloalkyl group, and an optionally substituted phenyl group,
or Y¹ and W¹ are tethered to form a cyclic unit, said tether being a C₃-C₆ alkanediyl chain with one or more heteroatoms or an unsaturated C₃-C₆ chain;
or Y² and W² are tethered to form a cyclic unit, said tether being a C₃-C₆ alkanediyl chain with one or more heteroatoms or an unsaturated C₃-C₆ chain;
R¹ and R², identical or different, are selected from the group consisting of: a C₁-C₆ alkyl group, a C₃-C₆ cycloalkyl, a C₆-C₁₀ aryl, and a (C₆-C₁₀)aryl(C₁-C₆)alkyl group, said C₁-C₆ alkyl group being optionally substituted with an hydroxyl group, or substituted with a —C(═O)—NH—(C₆-C₁₀)aryl, preferably with a —C(═O)—NH-phenyl group,
or R¹ and Y¹ can also be tethered to form a cyclic unit with the nitrogen atom bearing R¹, said tether being a C₃-C₄ alkanediyl chain or an unsaturated C₃-C₆ alkanediyl group, an heteroalkanediyl with one or more N atoms, wherein the carbons of the chain may also be part of a carbonyl group,
or R² and Y² can also be tethered to form a cyclic unit with the nitrogen atom bearing R², said tether being a C₃-C₄ alkanediyl chain or an unsaturated C₃-C₆ alkanediyl group, an heteroalkanediyl with one or more N atoms, wherein the carbons of the chain may also be part of a carbonyl group,
R³ and R′³ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R³ and R′³ together form a C₃-C₅alkanediyl chain, an unsaturated C₃-C₆ alkanediyl group, optionally substituted with a halo(C₁-C₆)alkyl such as CF₃, or an heteroalkanediyl group with O or N atoms, and
R⁴ and R′⁴ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R⁴ and R′⁴ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms.

2. A radiosensitizer comprising a monometallic (Amine)Platinum(II) N-Heterocyclic Carbene complex having the following formula (I-2): 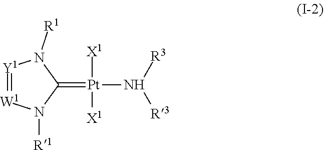
wherein:
R′¹ is selected from the group consisting of: a C₁-C₆ alkyl group, a C₃-C₆ cycloalkyl, a C₆-C₁₀ aryl, and a (C₆-C₁₀)aryl(C₁-C₆)alkyl group;
X¹ is selected from the group consisting of: iodide, bromide, chloride, and nitrato (ONO₂);
Y¹ is either a C—R⁵ group or a N atom, R⁵ being selected from the group consisting of: H, a C₁-C₆ alkyl group, C₃-C₆ cycloalkyl group, and an optionally substituted phenyl group,
W¹ is either a C—R⁶ group or a N atom, R⁶ being selected from the group consisting of: H, a C₁-C₆ alkyl group, C₃-C₆ cycloalkyl group, and an optionally substituted phenyl group,
or Y¹ and W¹ are tethered to form a cyclic unit, said tether being a C₃-C₆ alkanediyl chain with one or more heteroatoms or an unsaturated C₃-C₆ chain;
R¹ is selected from the group consisting of: a C₁-C₆ alkyl group, a C₃-C₆ cycloalkyl, a C₆-C₁₀ aryl, and a (C₆-C₁₀)aryl(C₁-C₆)alkyl group,
or R¹ and Y¹ can also be tethered to form a cyclic unit with the nitrogen atom bearing R¹, said tether being a C₃-C₄ alkanediyl chain or an unsaturated C₃-C₆ alkanediyl group, an heteroalkanediyl with one or more N atoms, wherein the carbons of the chain may also be part of a carbonyl group,
R³ and R′³ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R³ and R′³ together form a C₃-C₄ alkanediyl chain, an unsaturated C₃-C₆ alkanediyl group, or an heteroalkanediyl group with O or N atoms.

3. A bimetallic (Amine)Platinum(II) N-Heterocyclic Carbene complex having the following formula (I): 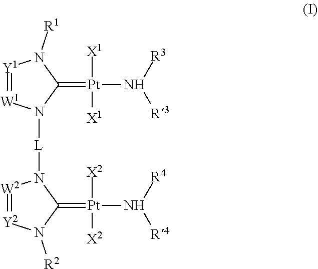
wherein:
L is a linker selected from the group consisting of: a C₁-C₁₂ alkanediyl group, a phenylene-bis(alkanediyl) group, a biphenyldiyl-bis(alkanediyl) group, and an heteroarylidene-bis(alkanediyl) group, said alkanediyl, phenylene and heteroarylidene groups being possibly substituted with one or several substituents such as C₁-C₆ alkyl groups, C₅-C₁₀ aryl groups, heteroaryl, and (hetero)cycloalkyl groups,
wherein said alkanediyl groups may be interrupted with one or several heteroatoms;
X¹ and X², identical or different, are selected from the group consisting of: iodide, bromide, chloride, and nitrato (ONO₂);
Y¹ and Y², identical or different, are either a C—R⁵ group or a N atom, R⁵ being selected from the group consisting of: H, a C₁-C₆ alkyl group, C₃-C₆ cycloalkyl group, and an optionally substituted phenyl group,
W¹ and W², identical or different, are either a C—R⁶ group or a N atom, R⁶ being selected from the group consisting of: H, a C₁-C₆ alkyl group, C₃-C₆ cycloalkyl group, and an optionally substituted phenyl group,
or Y¹ and W¹ are tethered to form a cyclic unit, said tether being a C₃-C₆ alkanediyl chain with one or more heteroatoms or an unsaturated C₃-C₆ chain;
or Y² and W² are tethered to form a cyclic unit, said tether being a C₃-C₆ alkanediyl chain with one or more heteroatoms or an unsaturated C₃-C₆ chain;
R¹ and R², identical or different, are selected from the group consisting of: a C₁-C₆ alkyl group, a C₃-C₆ cycloalkyl, a C₆-C₁₀ aryl, and a (C₆-C₁₀)aryl(C₁-C₆)alkyl group, said C₁-C₆ alkyl group being optionally substituted with an hydroxyl group, or substituted with a —C(═O)—NH—(C₆-C₁₀)aryl, preferably with a —C(═O)—NH-phenyl group,
or R¹ and Y¹ can also be tethered to form a cyclic unit with the nitrogen atom bearing R¹, said tether being a C₃-C₄ alkanediyl chain or an unsaturated C₃-C₆ alkanediyl group, an heteroalkanediyl with one or more N atoms, wherein the carbons of the chain may also be part of a carbonyl group,
or R² and Y² can also be tethered to form a cyclic unit with the nitrogen atom bearing R², said tether being a C₃-C₄ alkanediyl chain or an unsaturated C₃-C₆ alkanediyl group, an heteroalkanediyl with one or more N atoms, wherein the carbons of the chain may also be part of a carbonyl group,
R³ and R′³ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R³ and R′³ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms, and
R⁴ and R′⁴ are selected independently from the group consisting of: H, a C₁-C₈ alkyl, a C₃-C₆ cycloalkyl, a (C₆-C₁₀)aryl(C₁-C₆)alkyl, an optionally C₆-C₁₀ aryl, and a heterocycloalkyl group, or R⁴ and R′⁴ together form a C₃-C₅ alkanediyl chain, an unsaturated C₃-C₆ alkanediyl group, optionally substituted with a halo(C₁-C₆)alkyl such as CF₃, or an heteroalkanediyl group with O or N atoms.

4. The complex of claim 3, wherein, in formula (I), L is a C₂-C₁₂ alkanediyl group.

5. The complex of claim 3, wherein, in formula (I), R¹ and R² are C₁-C₆ alkyl groups.

6. The complex of claim 3, wherein, in formula (I), R³ or R′³ and R⁴ or R′⁴ are H or a cycloalkyl group.

7. The complex of claim 3, wherein, in formula (I), Y¹, Y², W¹ and W² are a CH group.

8. The complex of claim 3, having one of the following formulae:

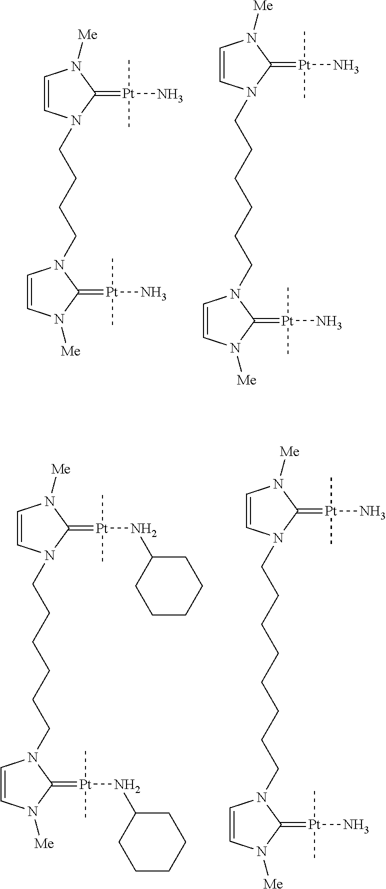

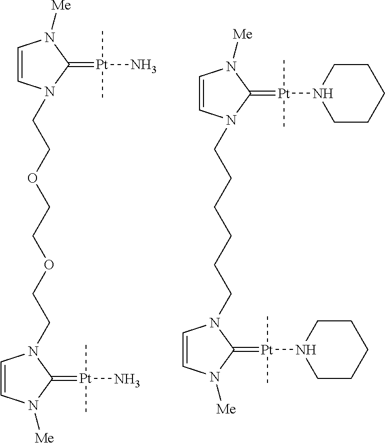

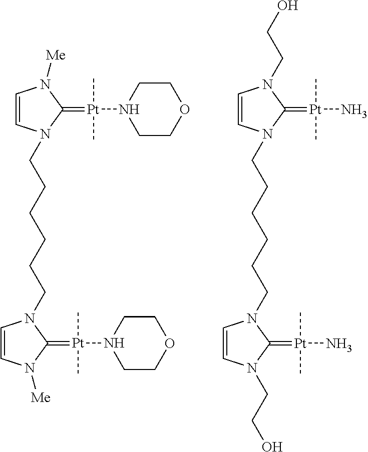

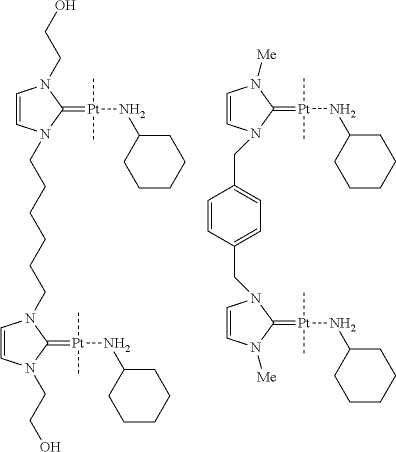

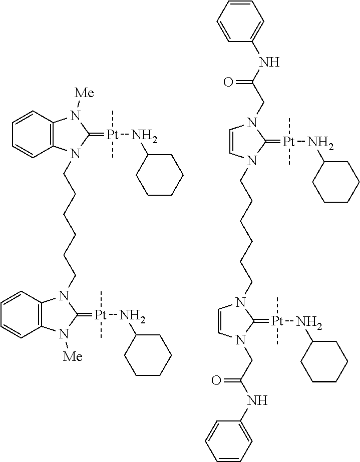

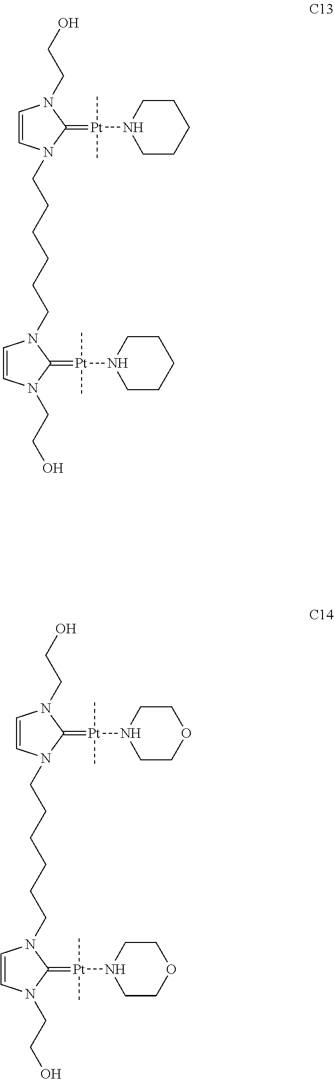

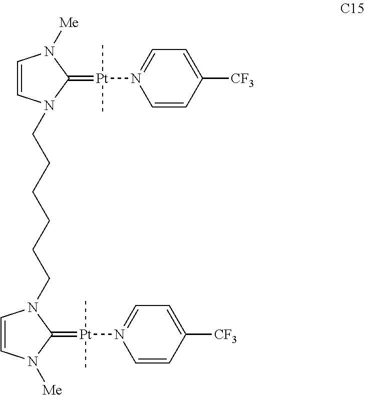

9. A complex having one of the following formulae:

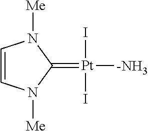

10. A conjugate comprising one or more complex(es) according to claim 3, covalently bound to at least one cell binding agent.

11. (canceled)

12. A medicament comprising a complex according to claim 3 or a conjugate comprising a plurality of the complexes covalently bound to at least one cell binding agent.

13. A pharmaceutical composition comprising a complex according to claim 3 or a conjugate comprising a plurality of the complexes covalently bound to at least one cell binding agent, and at least one pharmaceutically acceptable excipient.

14. A method for treating cancer comprising the administration of the complex of claim 3 or a conjugate comprising a plurality of the complexes covalently bound to at least one cell binding agent to a patient in need thereof.

15. A method for treating cancer comprising the administration to a patient in need thereof the complex of claim 3 or a conjugate comprising a plurality of the complexes covalently bound to at least one cell binding agent in combination with radiotherapy or in combination with radiotherapy and an anticancer drug.

16. The method of claim 14, wherein the cancer is selected from the group consisting of:
glioblastoma, lung cancer, non-small cell lung cancer, ovarian cancer, bladder cancer, rectal cancer, cervical cancer, and head and neck cancer.

17. The method of claim 15, wherein the cancer is selected from the group consisting of: glioblastoma, lung cancer, non-small cell lung cancer, ovarian cancer, bladder cancer, rectal cancer, cervical cancer, and head and neck cancer.
